(19) United States
(12) Patent Application Publication
(10) Pub. No.: US 2026/0102331 A1
(43) Pub. Date: Apr. 16, 2026

(54) TOPICAL SKIN CARE COMPOSITION FOR MODULATING EPIDERMAL GENE EXPRESSION

(71) Applicant: DERMA RESEARCH GROUP INC., Guelph (CA)

(72) Inventor: Dusan SAJIC, Guelph (CA)

(73) Assignee: DERMA RESEARCH GROUP INC., Guelph (CA)

(21) Appl. No.: 19/170,743

(22) Filed: Apr. 4, 2025

Related U.S. Application Data
(60) Provisional application No. 63/706,608, filed on Oct. 11, 2024.

(51) Int. Cl. A61K 8/9789; A61K 8/34; A61K 31/05; A61K 36/74; A61K 36/82; A61P 17/00; A61P 17/06; A61P 17/10; A61Q 19/08

(52) U.S. Cl. CPC A61K 8/9789; A61K 8/347; A61K 31/05; A61K 36/742; A61K 36/82; A61P 17/00; A61P 17/06; A61P 17/10; A61Q 19/08

(57) ABSTRACT

There is a topical skin care composition composed of a blend of hydroxytyrosol, green coffee extract and green tea leaf extract for use in the modulation of epidermal cellular gene expression profiles to alleviate various skin disorders and significantly reduce the resultant disease symptoms and reduce the rate at which the skin ages. More specifically, the topical skin care composition has hydroxytyrosol at a concentration range of 0.1% w/w to 0.75% w/w, green tea extract at a concentration range of 0.025% w/w to 0.1% w/w; and green coffee bean extract at a concentration range of 0.025% w/w to less than 0.2% w/w. A method of treating one or more of the hallmarks of ageing and skin degradation and collagen and elasticity as a single dosage from a pharmaceutical composition is described, as well as a method of activating skin cells having the step of applying the topical skin composition.

### CROSS-REFERENCE TO A RELATED APPLICATION

The present application claims priority based on U.S. Provisional Application No. 63/706,608, filed Oct. 11, 2024, which is incorporated herein by reference in its entirety.

### FIELD OF THE DISCLOSURE

This disclosure generally relates to skincare products, and more specifically to a topical skin care composition to reduce the presenting symptoms of various skin disorders through the regulation of cellular gene activity.

### DESCRIPTION OF THE RELATED ART

Maintenance of the skin and ensuring its proper health of the epidermis is a key component of an individual's general health. As the body's largest organ, the skin protects the individual external factors, such UV radiation, oxidative stress, viral infections, germs, and other factors that can directly affect the proper functioning/stasis of the skin. Exposure to such factors expedites the time-dependent change in the cell's functionality and reproducibility (process termed “premature cellular aging”). Skin aging is also a vital component of skin health. As skin cells age, the cells lose their proliferative capacity until such point where they enter a state of irreversible cell cycle arrest (the arrested state termed “senescence”).

Aging is a process defined as the time-dependent and persistent change in the functionality and reproducibility of cells within an organ or organism. Aging is directly correlated to an increased probability of morbidity and mortality. Within an organism, organs can age at varying rates, rates that are dependent upon and are directly impacted by exposure to various environmental stressors. An environmental stressor can dramatically increase the rate of aging which is directly correlated with the type, severity and duration of exposure to environmental stressors. It is not uncommon for an organism to exhibit organs with drastic variation in the rate of aging (expedited time-dependant change in cells) within a single organism.

Aging of the skin is caused by twelve hallmarks of aging (the “aging hallmarks”). Each hallmark affects the skin in a particular manner that results in the presentation of aged skin. First hallmark is loss of Proteostasis. Aging disrupts the balance of protein synthesis, folding and degradation, leading to dysfunctional proteins accumulating in the cells. Dysfunctional protein can accumulate in the cell thereby causing overload stress in the system and limiting active protein from reaching its target. The second hallmark is cellular senescence. Senescent cells cease to divide and begin to secrete inflammatory factors, which is a main contributing factor to tissue aging. The third hallmark is genomic instability. Accumulating of DNA damage leads to genomic instability, which results improper or dysfunctional protein synthesis and subsequent accelerating aging. Fourth hallmark of aging is telomere attrition. Shortening of telomeres leads to reduced regenerative capacity and thus cellular aging. The fifth hallmark is epigenetic alterations. Changes in gene expression regulation contributes to aging. Over expression or under expression of key protein factors can have detrimental effects on the cell, thereby aging the cell. The sixth hallmark is mitochondrial dysfunction. Impaired mitochondria reduce energy production and increase oxidative stress, both leading to cellular aging. The seventh hallmark is deregulated nutrient sensing. Aging affects pathways that sense and respond to nutrients, which disrupts metabolism. The eight hallmark of aging is stem cell exhaustion. Depletion of stem cell reserves reduces tissue regeneration causing cellular aging. The ninth hallmark is altered intercellular communication. Aging disrupts cell signalling, leading to tissue dysfunction and cellular aging. The tenth hallmark of aging is chronic inflammation. Persistent low-grade inflammation accelerates aging. The eleventh hallmark is Oxidative stress which is caused by reactive oxygen species (“ROS”). Excessive ROS damages cellular components, promoting aging. The twelfth hallmark is impaired autophagy. Reduced autophagy hinders the removal of damaged cellular components that exhaust the system and cause the cells to age.

Similar to, and often interconnected to skin aging, skin disease is often correlated to a change in the cellular activity as a result of an external factor or a change in the cellular pathway. The effects of skin disease can be devastating to the individual as the skin is an outer representation of the individual's health. The skin has many maintenance factors to prevent or retard the disease progression. These maintenance factors are modulated through cellular sensing or signalling pathways. It is advantageous for a Topical Skin Care Composition to affect these internal factors to upregulate its action, thereby increasing the ability to prevent or retard the disease progression.

The proper maintenance of the skin is achieved through innate/adaptive mechanisms (the “Cellular Responses”) as well as behavioural modifications (the “Behavioural Response(s)”). The Cellular Responses includes: (i) the skins own mechanism of maintaining the proper functioning and (ii) cellular responses to changes or external pressures within the dermis or the environment. Behavioural Response includes the individual's behaviour in maintaining their own skin.

Proper functioning of the skin is essential to the health of the individual. As the body's first contact with the external environment, the skin adapts to regulate its functioning to ensure that the skin can adapt and overcome external pressures and various attacks. The skin, as an organ, has several mechanisms to maintain its proper stasis and health-the innate and adaptive mechanisms. If these mechanisms are deficient, fail, or are circumvented by various agents or mutations, the skin as an organ breaks down, and various diseases manifest and present symptoms on the surface of the skin.

The skin has a variety of adaption mechanisms (“defense strategies”) to effectively protect the skin and the internal organs against diverse external factors. These defense strategies are in part mediated by gene expression profiles of the individual skin cells (the “epidermal cells”). Gene expression profiles are modulated within the skin through the influence of various external factors, the accumulation of reactive species, or activation of upstream regulatory proteins. In the simplest context, as a result of excess exposure to stress (UV-radiation from the sun), the epidermal cells will increase the expression of a gene responsible for the production of melanin, upon such expression, the melanin will absorb the harmful UVA, UVB, and UVC light. If the UV-radiation is not absorbed, as a result of insufficient expression of melanin, the UV-radiation may damage cellular DNA, which may lead to gene mutations and the onset of skin cancer (“melanoma”).

In some individuals the defense strategies are highly muted (“affected state”). Such an affected state may be a result of, but not limited to, genetic makeup, expression profile, mutations, and age. An affected state may have high consequence to the individual as the skin may be highly susceptible to the manifestation of various diseases, breakdown of the skin barrier, and the potential onset of melanoma. Individuals who are in an affected state should engage in behavioral mechanisms to reduce the likelihood of the manifestation of diseases and the onset of melanoma.

Behavioural mechanisms are actions the individual can take to further their own skin's ability to overcome external pressures and attacks. Through the adjustment of the individual's day to day routine, the individual can reduce the number and severity of external pressures on the skin, thereby maintaining the skins proper stasis and health without the need to activate the skins defense strategies. Some behavioral mechanisms include the marketed reduction in exposure to harmful UV rays, use of antibacterial ointments, use of sunscreen protection, and use of skin care products containing various ingredients (“skin care elements”).

Many skin care products contain skin care elements that have been previously shown to be effective in treating the skin and alleviating symptoms associated with skin disease. Skin care elements are only helpful if such skin care element is provided in its active bioavailable form and is targeted within the epidermal cells where it can be effective. For example, collagen is a readily used skin care element in various skin care products. Collagen that is provided in most skin care products is not targeted to the cytoplasm of the epidermal cells and rests on the surface of the skin. At that location, its effects may be minimal as the epidermal cells are unable to use the collagen in a manner required for proper cell functioning (creation and subsequent secretion of type I and II collagen fibers). Furthermore, application of collagen as a product ingredient does not activate cellular mechanism for the transcription and subsequent regulation of collagen.

Three Product adaptogen ingredients that have been used in skin care products are: hydroxytyrosol; Green tea extract; and green coffee extract (collectively “Product Ingredients”). First, hydroxytyrosol is a phenolic compound present in the fruit and leaf of the olive (Olea Europaea L.) and is most commonly derived from hydrolysis of oleurpopein. Hydroxytyrosol is one of the main components of virgin olive oil, olive mill wastewater, and olive leaf extracts. It is characterized by its chemical formula of C₈H₁₀O₃. Hydroxytyrosol has been shown as a phytochemical with antioxidant properties found to have anti-inflammatory, antiviral, antibacterial and antifungal properties. A number of filed patent applications have described the benefits of hydroxytyrosol when used in combination with other elements. One specific benefit described for the use of hydroxytyrosol relates to the beneficial application as an antioxidant for the treatment of oxygen free radical associated diseases. United States patent application US 2013-0266545 describes a composition and method of wound healing and inhibiting necrosis.

Second, Green tea extract is a naturally occurring antioxidant. It is an effective means of boosting collagen production in the skin. Green tea extract has also shown to increase the firmness of the skin while improving redness, elasticity, and overall texture. In addition, green tea extract is a great moisturiser.

Finally, green coffee extract is a naturally occurring antioxidant. It is an effective means of boosting collagen production in the skin. Green coffee bean extract has also shown to increase the firmness of the skin while improving redness and overall texture.

While it has been generally accepted that the administration of skin care products containing skin care elements with antioxidant properties would be beneficial to promote the reduction of free radicals, their use has not been previously shown to act as a potent activator of cellular transcription factors to induce a change in the genetic expression of epithelial cells. Antioxidants have only shown their ability to reduce the potential of free radicals within the cells of the skin, which if left untreated could cause DNA damage. The reduction of free radicals reduces the probability of DNA damage (caused the free radicals) but does not affect the cellular defense strategies associated with repairing any damage already caused by the free radicals. Furthermore, the use of such skin care elements has not been previously shown to aid cells with an affected state to increase the production of defense strategies.

Various attempts have been made to describe the effects of the Product Ingredients on the skin. Prior publications such as U.S. Pat. No. 9,511,034 (Garrett); U.S. Pat. No. 11,266,594 (Florence); and U.S. Pat. No. 9,889,171 (Chevreau) provide such examples for the use of Product Ingredients for the management and treatment of the skin.

Garrett discloses a skin treatment system containing hydroxytryrosol for managing skin conditions ranging from scars, including keloids, to burns and wrinkles. Specifically, Garrett discloses a silicone containing elastomer components to promote wound healing and decreasing cure time. Garrett does not contemplate the use of the compound to modulate DNA repair mechanisms nor the use of the compound to prevent or counter melanoma or to increase the transcription of various genes essential for optimum cellular and skin function and aid in defense strategies or to reverse skin aging. In addition, the Garrett does not contemplate the use of green tea, and green coffee bean.

Florence discloses a topical skin care formulation comprising of plant extracts used for the treatment of skin conditions arising from exposure to pollution or free radicals. Florence discloses a formulation comprising of an effective amount of Diospyros mespiliformis leaf extract. The disclosed skin care formulation is designed to reduce oxidative damage in the skin. Florence does not describe the use of hydroxytyrosol, green tea extract, and green coffee extract for prevention of, or countering melanoma. Additionally, Florence does not disclose a topical skin care formulation for increased collagen synthesis, modulation of ECM remodelling, or modulation of cellular defense strategies through gene expression profiles.

Chevreau discloses topical compositions and methods for reducing oxidative stress through the use of Brassica Plant extract. Chevreau discloses the use of Brassica Plant extract and Camellia oleifera extract to improve the appearance and overall health of the skin. The topical application of such composition has shown to simulate Nfr2 target genes to reduce oxidative stress. Specifically, expression of catalase is shown to be highly expressed, which is a key antioxidant enzyme in the body's defense against oxidative stress. The skin has also been shown to increase in appearance, in part due to the increasing density of the collagen network in the dermis. The prior patent does describe a topical skin cream, containing plant extract that can activate genetic expression of catalase that can reduce oxidative stress and increase collagen formation, but it fails to use hydroxytyrosol, green coffee extract, or green tea leaf extract and fails to activate a number of genetic pathways essential for the rejuvenation of the skin and fails to describe the use of the compound in the prevention of, or countering melanoma.

As such, there is a need for a Topical Skin Care Composition that has a multifaceted mode of activating cellular transcription factors to increase gene transcription and subsequent cellular protein production. A Topical Skin Care Composition that can increase in gene transcription factors to improve and fortify cellular defense mechanisms, increases DNA repair molecules and upregulate upstream factors and to increase production of potent cellular factors essential for cellular health and reverse or reduce skin aging. A skin care composition that can modulate lar gene expression to activate cellular repair mechanisms associated with DNA damage and to activate DNA expression profiles essential to rebuilding cellular components. Such modulation to have a dual role: first to reduce the symptoms of a skin condition or disease infliction; and second, to reduce the probability of their occurrence. Indeed, there is a need for a Topical Skin Care Composition that can be used in a variety of skin care products to reduce the likelihood of occurrence of a variety of skin conditions and if already presenting, can dramatically reduce the resultant symptoms, thereby leaving the skin in its natural healthy stasis.

### SUMMARY OF THE DISCLOSURE

The present disclosure provides a Topical Skin Composition for the use in the reduction of or elimination of various skin disorders. The Topical Skin Composition functions through the modulation of epidermal cellular gene expression profiles and through the activation of cellular repair mechanisms to rebuild and fortify key cellular components and to restore the health and stasis of the skin. In doing so, the Topical Skin Composition is capable of two primary functions associated in the treatment of the skin: 1. reducing or eliminating the probability of the occurrence of skin disorders; and 2. reducing the severity of presenting skin disorders. The modulation of the epidermal cellular gene expression profiles allows the cells to modulate defense strategies through the regulation of innate and adaptive mechanisms. In addition, the Topical Skin Composition provides a mechanism for the skin cells to reverse the signs of aging and normalize/aid the epidermis in: the reduction of ROS in presence of stress; reduction of IL-6; increase and stabilization of HIF-1α; increase production of DDIT4 factor; modulation of angiogenesis and apoptosis factors; increase in collagen synthesis; and, modulation of ECM remodelling.

The Topical Skin Composition can be formulated in a topical skin care product (moisturizer, sunscreen, serum) to protect the skin from the occurrence of various skin disorders or to treat and reduce the symptoms or severity of already inflicted skin disorders.

In an aspect, the present disclosure provides a method of activating skin cells, the method comprising: providing a topical skin composition comprising: hydroxytyrosol at a concentration range of 0.1% w/w to 0.75% w/w; green tea extract at a concentration range of 0.025% w/w to 0.1% w/w; and, green coffee bean extract at a concentration range of 0.025% w/w to less than 0.2% w/w; and, topically applying the topical skin composition to skin.

In another aspect, the present disclosure provides a topical skin composition comprising: hydroxytyrosol at a concentration range of 0.1% w/w to 0.75% w/w; green tea extract at a concentration range of 0.025% w/w to 0.1% w/w; and, green coffee bean extract at a concentration range of 0.025% w/w to less than 0.2% w/w; and, wherein the topical skin composition is applied to a surface of epidermal cells to modulate cellular gene expression profiles essential for skin health, prevention of skin diseases and reduction of severity of affected skin cells.

In yet another aspect, the present disclosure provides a method of treating one or more of the hallmarks of ageing and skin degradation and collagen and elasticity as a single dosage from a pharmaceutical composition, the method comprising: providing the pharmaceutical composition comprising: hydroxytyrosol at a concentration range of 0.1% w/w to 0.75% w/w; green tea extract at a concentration range of 0.025% w/w to 0.1% w/w; and, green coffee bean extract at a concentration range of 0.025% w/w to less than 0.2% w/w; and, topically applying the pharmaceutical composition to skin.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

It will now be convenient to describe the disclosure with particular reference to one embodiment of the present disclosure. It will be appreciated that the figures relate to one embodiment of the present disclosure only and are not to be taken as limiting the disclosure.

FIG. 1 is a qPCR quantitative analysis graph of expressed P4H1 mRNA in epidermal cells. P4H1 mRNA is associated with collagen synthesis. Epidermal cells incubated with various creams or cream vehicles and were grown in the presence or absence of UV radiation. Epidermal cells were tested with Protectif Tinted and Renutriate Professional and their respective mRNA expression was compared to the epidermal cells treated with vehicles (vehicles being the Renutriate Professional and Protectif Tinted without containing the Product Ingredients). Cells treated with Renutriate Professional and Protectif Tinted were able to produce a greater than three (3) fold increase in P4H1 mRNA.

FIG. 2 is a qPCR quantitative analysis graph of expressed LAMB1 mRNA in epidermal cells. LABM1 mRNA associated with ECM remodeling. Epidermal cells were incubated with various creams or cream vehicles and were grown in the presence or absence of UV radiation. Epidermal cells were tested with Protectif Tinted and Renutriate Professional and their respective mRNA expression was compared to the epidermal cells treated with vehicles (vehicles being the Renutriate Professional and Protectif Tinted without containing the Product Ingredients). Cells treated with Renutriate Professional and Protectif Tinted were able to produce a greater than one and a half (1.5) fold increase in LAMB1 mRNA.

FIG. 3 is a qPCR quantitative analysis of expressed LAMB4 mRNA in epidermal cells. LABM4 mRNA associated with ECM remodeling. Epidermal cells were incubated with various creams or cream vehicles and were grown in the presence or absence of UV radiation. Epidermal cells were tested with Protectif Tinted and Renutriate Professional and their respective mRNA expression was compared to the epidermal cells treated with vehicles (vehicles being the Renutriate Professional and Protectif Tinted without containing the Product Ingredients). Cells treated with Renutriate Professional and Protectif Tinted were able to produce a greater than one and a half (1.5) fold increase in LAMB4 mRNA.

FIG. 4 is a qPCR quantitative analysis of expressed LAMA4 mRNA in epidermal cells. LABA4 mRNA associated with ECM remodeling. Epidermal cells were incubated with various creams or cream vehicles and were grown in the presence or absence of UV radiation. Epidermal cells were tested with Protectif Tinted and Renutriate Professional and their respective mRNA expression was compared to the epidermal cells treated with vehicles (vehicles being the Renutriate Professional and Protectif Tinted without containing the Product Ingredients). Cells treated with Renutriate Professional and Protectif Tinted were able to produce a greater than one and a half (1.5) fold increase in LAMA4 mRNA.

FIG. 5 is a qPCR quantitative analysis of expressed DCN mRNA in epidermal cells. DCN mRNA associated with ECM remodeling. Epidermal cells were incubated with various creams or cream vehicles and were grown in the presence or absence of UV radiation. Epidermal cells were tested with Protectif Tinted and Renutriate Professional and their respective mRNA expression was compared to the epidermal cells treated with vehicles (vehicles being the Renutriate Professional and Protectif Tinted without containing the Product Ingredients). Cells treated with Renutriate Professional and Protectif Tinted were able to produce a greater than one and a half (1.5) fold increase in DCN mRNA.

FIG. 6A is a qPCR quantitative analysis of expressed IL6 mRNA in epidermal cells. IL6 mRNA associated with stress and inflammatory response. Epidermal cells were incubated with various creams or cream vehicles and were grown in the presence or absence of UV radiation. Skin cells were tested with Protectif Tinted and Renutriate Professional and their respective mRNA expression was compared to the epidermal cells treated with vehicles (vehicles being the Renutriate Professional and Protectif Tinted without containing the Product Ingredients). Cells treated with Renutriate Professional and Protectif Tinted were able to produce a greater than two (2) fold decrease in IL6 mRNA.

FIG. 6B is a protein quantitative analysis of expressed IL6 protein in epidermal cells. IL6 protein is associated with stress and inflammatory response. Epidermal cells were incubated with various creams or cream vehicles and were grown in the presence or absence of UV radiation. Epidermal cells were tested with Protectif Tinted and Renutriate Professional and their respective protein expression was compared to the epidermal cells treated with vehicles (vehicles being Renutriate Professional and Protectif Tinted without containing the Product Ingredients). Cells treated with Renutriate Professional and Protectif Tinted were able to reduce the production of IL6 by 125 pg/ml than the respective vehicles.

FIG. 7A is a qPCR quantitative analysis of expressed IL1b mRNA in epidermal cells. IL1b mRNA associated with stress and inflammatory response. Epidermal cells were incubated with various creams or cream vehicles and were grown in the presence or absence of UV radiation. Epidermal cells were tested with Protectif Tinted and Renutriate Professional and their respective mRNA expression was compared to the epidermal cells treated with vehicles (vehicles being the Renutriate Professional and Protectif Tinted without containing the Product Ingredients). Cells treated with Renutriate Professional and Protectif Tinted were able to produce a greater than three (3) fold decrease in IL1b mRNA.

FIG. 7B is a protein quantitative analysis of expressed IL1b protein in epidermal cells. IL1b protein is associated with stress and inflammatory response. Epidermal cells were incubated with various creams or cream vehicles and were grown in the presence or absence of UV radiation. Epidermal cells were tested with Protectif Tinted and Renutriate Professional and their respective protein expression was compared to epidermal cells treated with the vehicles (vehicles being Renutriate Professional and Protectif Tinted without containing the Product Ingredients). Cells treated with Renutriate Professional and Protectif Tinted were able to reduce the production of IL1b by 60 pg/ml than the respective vehicles.

FIG. 8 is a qPCR quantitative analysis of expressed DDIT4 mRNA in epidermal cells. DDIT4 mRNA associated with stress and inflammatory response. Epidermal cells were incubated with various creams or cream vehicles and were grown in the presence or absence of UV radiation. Epidermal cells were tested with Protectif Tinted and Renutriate Professional and their respective mRNA expression was compared to the epidermal cells treated with vehicles (vehicles being the Renutriate Professional and Protectif Tinted without containing the Product Ingredients). Cells treated with Renutriate Professional and Protectif Tinted were able to produce a greater than one and a half (1.5) fold increase in DDIT4 mRNA.

FIG. 9 is a qPCR quantitative analysis of expressed VEGF mRNA in epidermal cells. VEGF mRNA associated with angiogenesis. Epidermal cells were incubated with various creams or cream vehicles and were grown in the presence or absence of UV radiation. Epidermal cells were tested with Protectif Tinted and Renutriate Professional and their respective mRNA expression was compared to the epidermal cells treated with vehicles (vehicles being the Renutriate Professional and Protectif Tinted without containing the Product Ingredients). Cells treated with Renutriate Professional and Protectif Tinted were able to produce a greater than two (2) fold increase in VEGF Mrna.

FIG. 10 is a qPCR quantitative analysis of expressed BNIP3 mRNA in epidermal cells. BNIP3 mRNA associated with apoptosis. Epidermal cells were incubated with various creams or cream vehicles and were grown in the presence or absence of UV radiation. Epidermal cells were tested with Protectif Tinted and Renutriate Professional and their respective mRNA expression was compared to the epidermal cells treated with vehicles (vehicles being the Renutriate Professional and Protectif Tinted without containing the Product Ingredients). Cells treated with Renutriate Professional and Protectif Tinted were able to reduce BNIP3 mRNA by a greater than three (3) fold decrease.

FIG. 11 is a protein quantitative analysis of expressed HIF-1α in epidermal cells. HIF-1α protein is associated with cellular stress and presence of oxidative reactive species. Epidermal cells were incubated with various creams or cream vehicles and were grown in the presence or absence of UV radiation. Epidermal cells were tested with Protectif Tinted and Renutriate Professional and their respective protein expression was compared to the epidermal cells treated with vehicles (vehicles being Renutriate Professional and Protectif Tinted without containing the Product Ingredients). Cells treated with Renutriate Professional and Protectif Tinted were able to produce 50 pg/ml more HIF-1α protein than the respective vehicles.

FIG. 12 is a quantitative analysis of ROS production in epidermal cells. ROS is associated with oxidative stress which can lead to cellular damage (lipid peroxidation and DNA fragmentation). Epidermal cells were incubated with various creams or cream vehicles and were grown in the presence or absence of UV radiation. Epidermal cells were tested with Protectif Tinted and Renutriate Professional and their respective protein expression was compared to the epidermal cells treated with vehicles (vehicles being Renutriate Professional and Protectif Tinted without containing the Product Ingredients). Cells treated with Renutriate Professional and Protectif Tinted were able to decrease the amount of normalized ROS by 200 units in presence of UV when compared to their respective vehicles. As expected, there was no increase in ROS without the presence of UV.

FIGS. 13A to 13E are images of a patient's head (Patient 1) while using Renutriate PRO product. Patient 1 would use a small amount of Renutriate PRO twice (2) a day for eight (8) months.

FIG. 13A is the patient's skin at day zero (0).

FIG. 13B is the patient's skin upon continued use of Renutriate PRO at day sixty (60). FIG. 13C is the patient's skin upon continued use of Renutriate PRO at day one hundred and twenty (120).

FIG. 13D is the patient's skin upon continued use of Renutriate PRO at day one hundred and eighty (180).

FIG. 13E is the patient's skin displays a rejuvenated and healthy appearance after two-hundred and forty (240) days of continued use of Renutriate PRO.

FIG. 14A is the patient's skin at day zero (0) upon continued use of Renutriate PRO at day two-hundred and forty (240). FIG. 14 is an image of a patient's face (Patient 2) while using Renutriate PRO product. Patient 2 would use a small amount of Renutriate PRO twice (2) a day for four (4) months.

FIG. 14B is the patient's skin upon continued use of Renutriate PRO at day sixty (60).

FIG. 14C is the patient's skin upon continued use of Renutriate PRO at day one hundred and twenty (120);

FIGS. 15A to 15C are images of a patient's face (Patient 3) while using Renutriate PRO product. Patient 3 would use a small amount of Renutriate PRO twice (2) a day for four (4) months.

FIG. 15A is the patient's skin at day zero (0).

FIG. 15B is the patient's skin upon continued use of Renutriate PRO at day sixty (60).

FIG. 15C is the patient's skin upon continued use of Renutriate PRO at day one hundred and twenty (120).

FIGS. 16A and 16B are images of a patient's face (Patient 4) while using Renutriate PRO product. Patient 4 would use a small amount of Renutriate PRO twice (2) a day for two (2) months.

FIG. 16A is the patient's skin at day zero (0).

FIG. 16B is the patient's skin upon continued use of Renutriate PRO at day sixty (60).

FIGS. 17A and 17B are images of a patient's leg (Patient 5) while using Renutriate PRO product. Patient 5 would use a small amount of Renutriate PRO twice (2) a day for two (2) months.

FIG. 17A is the patient's skin at day zero (0).

FIG. 17B is the patient's skin upon continued use of Renutriate PRO at day sixty (60);

FIGS. 18A and 18B are images of a patient's shoulders (Patient 6) while using Renutriate PRO product only on the left shoulder. Patient 6 used a standard over the counter moisturizer on the right shoulder. Patient 6 would use a small amount of Renutriate PRO on the left shoulder twice a day for two weeks (14 days) after patient 6 sustained a mild sunburn.

FIG. 18A is an image of the patient's left shoulder after using Renutriate PRO for two weeks (14 days) post mild sunburn.

FIG. 18B is an image of the patient's right shoulder after using a standard over the counter moisturizer for two weeks (14 days) post mild sunburn.

FIGS. 19A to 19C of a patient's face (Patient 7) while using Renutriate product. Patient 7 would use a small amount of Renutriate twice (2) a day for sixteen (16) weeks.

FIG. 19A is the patient's skin at day zero (0).

FIG. 19B is the patient's skin upon continued use of Renutriate at eight (8) weeks.

FIG. 19C is the patient's skin upon continued use of Renutriate at sixteen (16) weeks.

FIGS. 20A to 20C are images of a patient's hand (Patient 8) while using Renutirate product. Patient 8 would use a small amount of Renutriate twice (2) a day for two (2) weeks.

FIG. 20A is the patient's skin at day zero (0).

FIG. 20B is the patient's skin upon continued use of Renutriate at one (1) week.

FIG. 20C is the patient's skin upon continued use of Renutriate at two (2) weeks.

FIGS. 21A to 21D are images of a patient's ankle (Patient 9) while using Renutirate product. Patient 9 would use a small amount of Renutriate twice (2) a day for sixteen (16) weeks.

FIG. 21A is the patient's skin at day zero (0).

FIG. 21B is the patient's skin upon continued use of Renutriate at eight (8) weeks.

FIG. 21C is the patient's skin upon continued use of Renutriate at twelve (12) weeks.

FIG. 21D is the patient's skin upon continued use of Renutriate at sixteen (16) weeks.

FIGS. 22A to 22C 22 is an image of a patient's hand (Patient 10) while using Renutirate product. Patient 10 would use a small amount of Renutriate twice (2) a day for sixteen (16) weeks.

FIG. 22A is the patient's skin at day zero (0).

FIG. 22B is the patient's skin upon continued use of Renutriate at eight (8) weeks.

FIG. 22C is the patient's skin upon continued use of Renutriate at 16 weeks.

FIGS. 23A to 23C are images of a patient's face (Patient 11) while using Renutirate product. Patient 11 would use a small amount of Renutriate twice (2) a day for sixteen (16) weeks.

FIG. 23A shows the patient's face at day zero (0).

FIG. 23B is the patient's skin upon continued use of Renutriate at eight (8) weeks.

FIG. 23C is the patient's skin upon continued use of Renutriate at 16 weeks.

FIGS. 24A and 24B are a quantitative skin age analysis review and ultrasound of patient's left and right forearm (Patient 12). Patient 12 has been applying the Renutriate PRO product on the left forearm once a day for nine (9) months. Patient 12 has been applying a leading brands product on the right forearm once a day for nine (9) months.

FIG. 24A is the skin age assessment of Patient 12's left forearm.

FIG. 24B is the skin age assessment of Patient 12's right forearm.

FIGS. 25A to 25C are a quantitative skin age analysis review of patient's arm treated with either Renutirate PRO product or leading brand product for 20 minutes and subsequently tested with UVB exposure (Patient 13).

FIG. 25A is a visual representation of Patent 13's arm and location of treatment prior to UVB exposure.

FIG. 25B is the skin age assessment of Patient 13's skin that was treated with Renutriate PRO.

FIG. 25C is the skin age assessment of Patient 13's skin that was treated with the leading brand product.

FIGS. 26A and 26B are a quantitative skin age analysis review of patient 14's face 90 min post application of Renutriate PRO product.

FIG. 26A is the skin age assessment of Patient 14's face prior to the application of the Renutriate PRO product.

FIG. 26B is the skin age assessment of Patient 14's face post treatment with the Renutriate PRO product.

### DETAILED DESCRIPTION OF THE DISCLOSURE

The present disclosure will now be described more fully hereinafter with reference to the accompanying images, in which preferred and other embodiments of the disclosure are shown. No embodiment described below limits any claimed disclosure and any claimed disclosure may cover processes or element that are not described below. The claimed disclosures are not limited to products or elements having all the features of any one element or process described below or to features common to multiple or all of the elements described below. It is possible that an element or process described below is not an embodiment of any claimed disclosure. The applicants, inventors or owners reserve all rights that they may have in any disclosure claimed in this document, for example the right to claim such disclosure in a continuing application and do not intend to abandon, disclaim or dedicate to the public any such disclosure by its disclosure in this document.

A novel Topical Skin Composition for use in the treatment of skin disorders and various auto immune disease has now been developed with significant advantages over pre-existing medications or products. In particular, it has been determined that the use of the Topical Skin Composition of the present disclosure can effectively modulate gene transcription factors of various key modulators of defense strategies in the epidermal cells. In doing so, the individual cells of the epidermis are able to effectively reduce disease expression and significantly reduce the probability of a such disease from forming. In addition, the modulation of gene expression has been shown to effectively reduce the occurrence and severity of various autoimmune diseases. Finally, the modulation of gene expression has been shown to reverse and retard the signs of skin aging.

Overall, the present skin care composition is to reduce the presenting symptoms of various skin disorders through the regulation of cellular gene activity. Such presenting symptoms have been shown to be detrimental on the health of the skin. As a result of a decrease in the health of the skin, the skin loses its resiliency allowing skin disorders to manifest and the skin to begins to age. Through the modulation of epidermal cellular gene expression and the activation of cellular repair mechanisms, the Topical Skin Composition dramatically increases the total amount of key cellular components which restores the skin to its natural healthy stasis while also fortifying the skins natural repair and defense mechanisms. Restoring the skin to its natural healthy state while also increasing its defense mechanisms aids in rejuvenating the skin making it healthier and look younger. The Topical Skin Composition relates to the following effects on the epidermis: the reduction of ROS in presence of UV; reduction of Interleukin (IL)-6; increase and stabilization of HIF-1α as a result of hypoxia-responsive elements; increase in production of DDIT4; modulation of Angiogenesis and Apoptosis factors; increase in collagen synthesis; and, modulation of ECM remodelling. More specifically, the present disclosure relates to a unique blend of cosmeceutical agents to harnesses the synergistic effects of multiple natural compounds to deliver enhanced therapeutic benefits. The disclosure details the composition of the blend, the method of application, and the observed clinical outcomes, supporting its efficacy in dermatological applications.

The Product Ingredients act together to modulate cellular biological processes to protect the skin from external factors, autoimmune diseases, and age-related issues. Such effects allow the Product Ingredients to address the hallmarks of aging, providing a comprehensive approach to maintaining skin health and restoring skin to its natural healthy state. By stimulating collagen production, reducing inflammation, promoting cellular repair, and enhancing stress response, Product Ingredients of the present disclosure are specifically formulated to counteract the detrimental effects of environmental stressors (such as UV exposure), inflammation and autoimmune disorders. The present disclosure is described as using hydroxytyrosol, but a worker skilled in the relevant would appreciate that hydroxytyrosol is derived from Olea europaea, and any derivative thereof could be used as an alternative.

The Product Ingredients of the present disclosure have been shown to affect protein production of key cellular components within the epidermal cells. Such gene expression modulation has beneficial effects for the skin, including but not limited to protection from various external factors, disease progression, severity of disease, and maintenance of skin stasis. Gene expression in cells can be seen in one of two ways: mRNA expression (qPCR assay); and Protein Production (ELISA assay).

The Product Ingredients can be formulated into a variety of topical skin care products, including moisturizers, sunscreens, and serums. The Product Ingredients for the present disclosure have been formulated in moisturizer variants presently termed “Renutriate” (Restorative, Advanced, Professional) and sunscreens variants presently termed “Protectif” (untinted, fair tint, deep tint), and Rejuvenat. A worker skilled in the relevant art would appreciate that the Product Ingredients can be used, or formulated in any other product, including ingestible supplements. For the determination of the effects of Product Ingredients, the Renutriate Professional variant (“Renutriate PRO”), Renutriate Restorative (“Renutriate”) and Protectif Fair Tint (“Protectif Tint”) have been tested in vitro on skin cells and compared to skin cells treated with vehicles of each product (each vehicle being the identical corresponding product lacking the Product Ingredients). The cells were then grown in the absence of UV radiation or in presence of UV radiation and subsequently tested for gene and protein expression profiles.

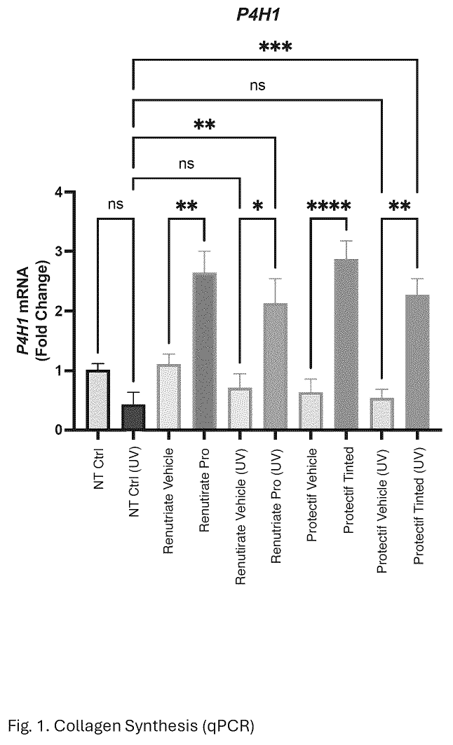

As further described in FIG. 1, epidermal cells were treated with the cream products containing the Product Ingredients or the Vehicle and tested in the presence or absence of UV radiation (stress). Product Ingredients in Renutriate PRO and Protectif Tinted significantly increased collagen mRNA production under stress (e.g., UV exposure) compared to the vehicle, as evidenced by P4H1 gene expression. P4H1 (Prolyl 4-hydroxylase subunit alpha-1) plays a key role in collagen synthesis, essential for maintaining skin's structural integrity. Even in absence of UV, cells treated with the Product Ingredients were able to produce significantly higher amount of P4H1. Product Ingredients stimulation of P4H1 enhances collagen biosynthesis, which counteracts collagen loss caused by aging and environmental stress. The increased collagen caused by Product Ingredients leads to firmer, more elastic skin and reduced wrinkles with skin sagging and hallowing around the eyes and face. Furthermore, loss of collagen makes the skin more fragile and increases aging.

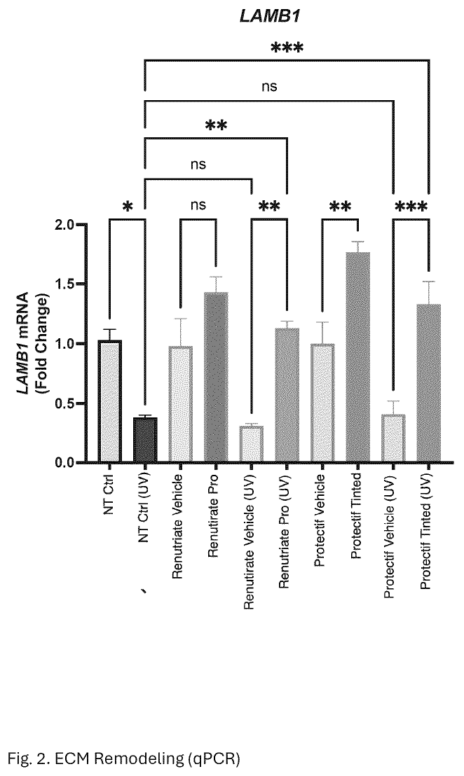

As further described in FIG. 2, the epidermal cells were treated with the cream products containing the Product Ingredients or the Vehicle and tested in the presence or absence of UV radiation (stress). Product Ingredients in Renutriate PRO and Protectif Tinted significantly increased the production of Lamin B1 (LAMB1) mRNA under stress (e.g., UV exposure) compared to the vehicle. LAMB1 is critical for maintaining nuclear integrity and chromatin organization. An increased expression of LAMB1 is critical for optimal epidermal function. LAMB1 is essential for maintaining nuclear integrity and chromatic organization. A decrease in cellular LAMB1 is associated with cellular senescence, a phenomenon characterized by the cessation of cell division. Cells with low LAMB1 are characterized by a permanent proliferation arrest and in response to endogenous and exogenous stress, including but not limited to, telomere dysfunction, oncogene activation and persistent DNA damage. Cellular senescence is also associated with aging and contributes directly to impaired tissue regeneration and chronic age-related diseases. By increasing expression of LAMB1 the Product Ingredients help in maintaining genomic stability, reduces the onset of cellular dysfunction, and dramatically reduces the chances of cancer formation. Furthermore, through increase in LAMB1 within the epidermal cells, the Product Ingredients limit the aging process and maintain the appearance of younger looking skin.

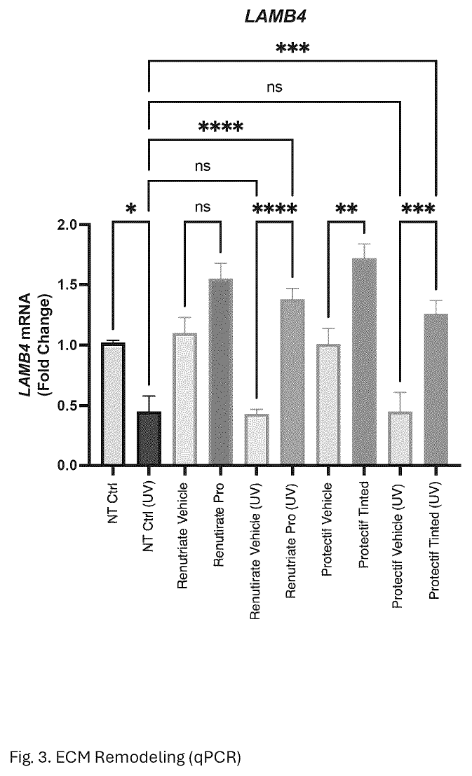

As further described in FIG. 3, the epidermal cells were treated with the cream products containing the Product Ingredients or the Vehicle and tested in the presence or absence of UV radiation (stress). The Product Ingredients in Renutriate PRO and Protectif Tinted significantly increased the production of Lamin B4 (LAMB4) mRNA under stress (e.g., UV exposure) compared to the vehicle. An increased expression of LAMB4 is critical for ECM remodeling and enhancing skin elasticity. LAMB4 functions in conjunction with LAMB1 to support nuclear structure and cellular proliferation. The Product Ingredients, by increasing the LAMB4 expression, enhances ECM remodeling, maintains skin elasticity and keeps the skin looking young and rejuvenated. One benefit of increased LAMB4 is maintaining skin elasticity and preventing from skin sagging. The increase in LAMB4 within the epidermis supports the maintenance of nuclear integrity thereby addressing the hallmarks of loss of proteostasis and genomic instability. By increasing nuclear integrity and maintaining skin elasticity, the Product Ingredients reduce the signs of aging and maintain a rejuvenated appearance to the skin.

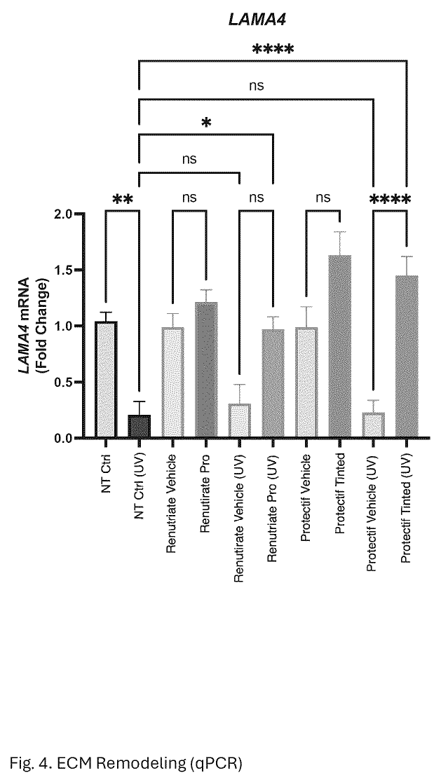

As further described in FIG. 4, the epidermal cells were treated with the cream products containing the Product Ingredients or the Vehicle and tested in the presence or absence of UV radiation (stress). The Product Ingredients in Renutriate PRO and Protectif Tined significantly increased the production of Lamin A4 (LAMA4) mRNA under stress (e.g., UV exposure) compared to the vehicle. An increased expression of LAMA4 is critical for maintaining nuclear architecture and effective gene regulation. LAMA4 contributes to epidermal nuclear integrity and regulation of key genetic modulators involved in cellular repair and collagen production. By enhancing LAMA4 expression, the Product Ingredients strengthen and fortify the skin's natural repair mechanism and ensuring better resilience to agung. The upregulation of LAMA4 by the Protein Ingredients addresses the hall mark of epigenetic alterations through proper gene expression. Product Ingredients maintains proper gene expression and prevents premature cellular aging.

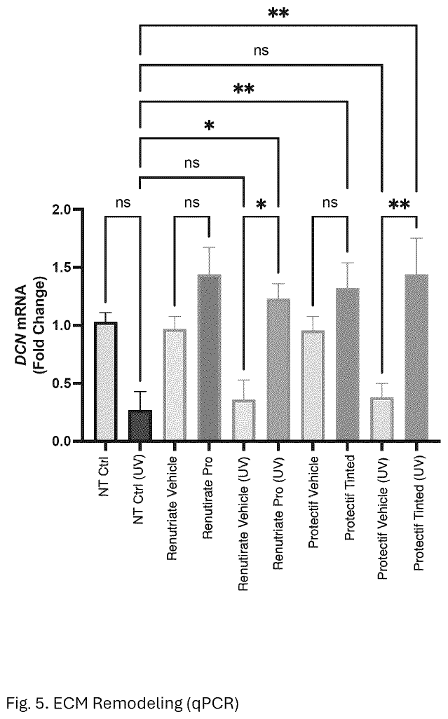

As further described in FIG. 5, the epidermal cells were treated with cream products containing the Product Ingredients or the Vehicle and tested in the presence or absence of UV radiation (stress). The Product Ingredients in Renutriate PRO and Protectif Tinted significantly increased the production of Decorin (“DCN”) mRNA under naïve and in stress conditions (e.g., UV exposure) compared to the Vehicle. An increased expression of DCN assists in collagen fiber assembly and ECM stability while also ensuring proper collagen organization via fibrillogenesis. DCN is the main proteoglycan in the skin and regulates the collagen matrix assembly. DCN is an important constituent of the skin connective tissue and are essential for maintaining mechanical strength of the skin. The upregulation of DCN levels by Product Ingredients results in smoother, firmer skin. DCN prevents extracellular matrix deterioration and directly address aging-related tissue fragility. Degradation or loss of DCN protein in aged skin (age-dependent alteration) contributes to skin fragility, which is often seen in elderly people. DCN also modulates epidermal growth factors like TGF-β, further influencing cell growth and differentiation.

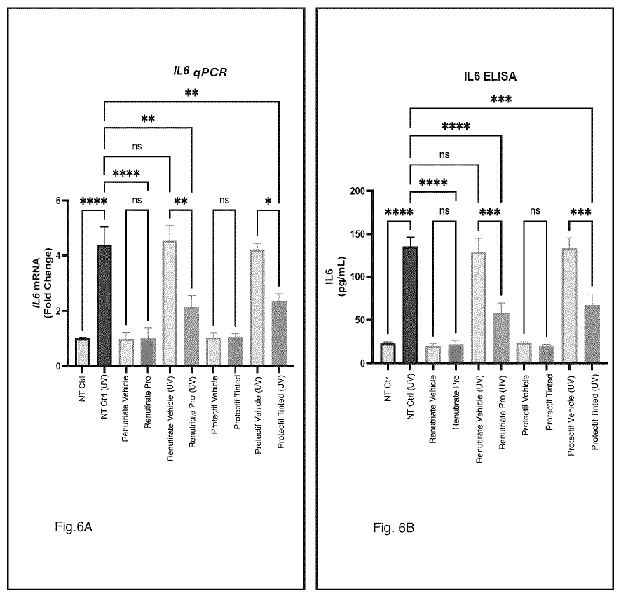

As further described in FIG. 6, the epidermal cells were treated with cream products containing the Product Ingredients or the Vehicle and tested in the presence or absence of UV radiation (stress). The Product Ingredients in Renutriate PRO and Protectif Tinted significantly reduced the production of IL-6, a key inflammation marker under stress conditions (e.g., UV exposure) compared to Vehicle. FIG. 6a assesses the presence of IL-6 mRNA within the cells, while FIG. 6b assess the presence of IL-6 protein within the cells. The amount of mRNA within the epidermal cells correlated with the presence of IL-6 protein. IL-6 is a pro-inflammatory cytokine that contributes to chronic inflammation. Reduction of inflammation by Product Ingredient is key to the prevention of long-term skin damage caused by stress. Furthermore, chronic inflammation accelerates aging by contributing to tissue breakdown.

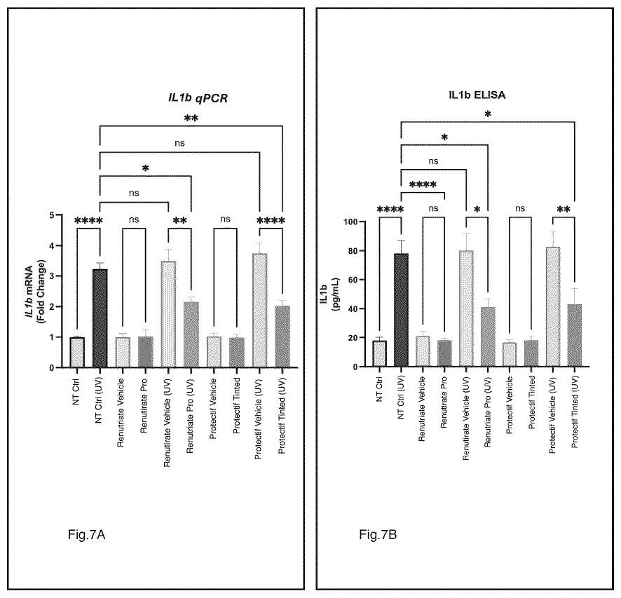

As further described in FIG. 7, epidermal cells were treated with cream products containing the Product Ingredients or the Vehicle and tested in the presence or absence of UV radiation (stress). The Product Ingredients in Renutriate PRO and Protectif Tinted significantly reduced the production of IL-16, a key inflammation marker in stress conditions (e.g., UV exposure). FIG. 7A assesses the presence of IL-13 mRNA within the cells, while FIG. 6B assess the presence of IL-16 protein within the cells. The amount of IL-13 mRNA within the cells correlated with the presence of IL-13 protein within the cells. IL-16 drives skin inflammation and irritation, often leading to collagen breakdown. Reduction of IL-13 by Product Ingredients reduces inflammation, protecting the skin from premature cellular aging and promoting a calm, healthy environment for skin regeneration. Inflammation is a hallmark of aging, and reduction of IL-13 improves skin health and skin longevity.

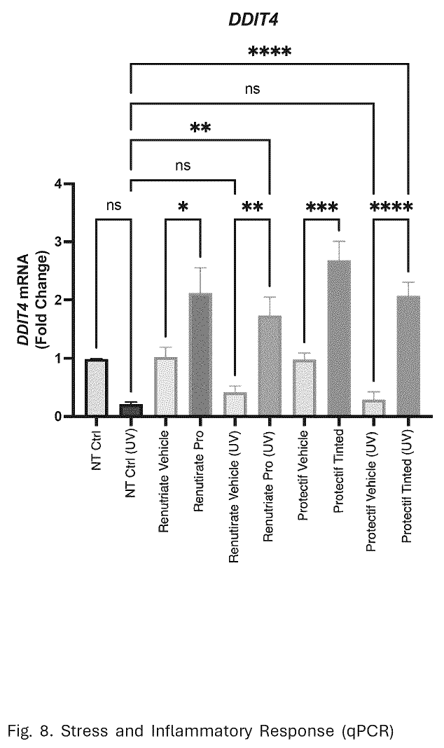

As further described in FIG. 8, epidermal cells were treated with cream products containing the Product Ingredients or the Vehicle and tested in the presence or absence of UV radiation (stress). The Product Ingredients in Renutriate PRO and Protectif Tinted significantly increased the production of DDIT4 mRNA in naïve conditions and under stress conditions (e.g., UV exposure) compared to Vehicle. Increase in DDIT4 expression promotes cellular resilience to stress, particularly UV-induced damage. DDIT4 protein is involved in regulating the mTOR pathway, which controls cellular metabolism and stress responses. DDIT4 is a key component in DNA repair mechanism necessary to repair DNA damage or DNA breaks caused by UV-radiation. Increase in DDIT4 expression and protein production by Protein Ingredient ensures that skin cells are better equipped to handle environmental challenges, including UV radiation, promoting repair and recovery. The ability to manage cellular stress is critical for preventing damage accumulation. Through increase production of DDIT4, the Product Ingredient greatly limits genomic instability and mitochondrial dysfunction, all key hallmarks of aging.

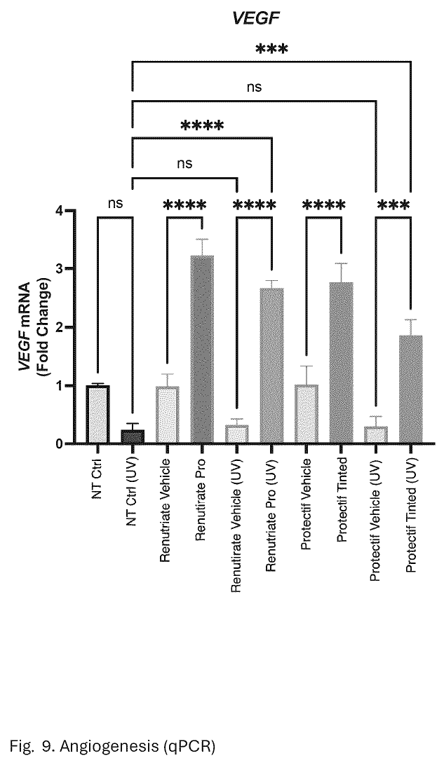

As further described in FIG. 9, epidermal cells were treated with cream products containing the Product Ingredients or the Vehicle and tested in the presence or absence of UV radiation (stress). The Product Ingredients in Renutriate PRO and Protectif Tinted significantly increased the production of VEGF mRNA in naïve conditions and under stress conditions (e.g., UV exposure) compared to Vehicle. Stimulation of VEGF expression promotes the formation of new blood vessels which in turn improves skin radiance. VEGF is critical for stimulating angiogenesis, ensuring that skin receives adequate blood supply and oxygenation. The Product Ingredients, by stimulating VEFG expression, enhances skin healing and rejuvenation, particularly and damage from environmental factors such as UV-radiation exposure. VEGF upregulation combats mitochondrial dysfunction by improving oxygen delivery and supporting energy production of the epidermal cells, facilitating faster recovery and enhanced radiance.

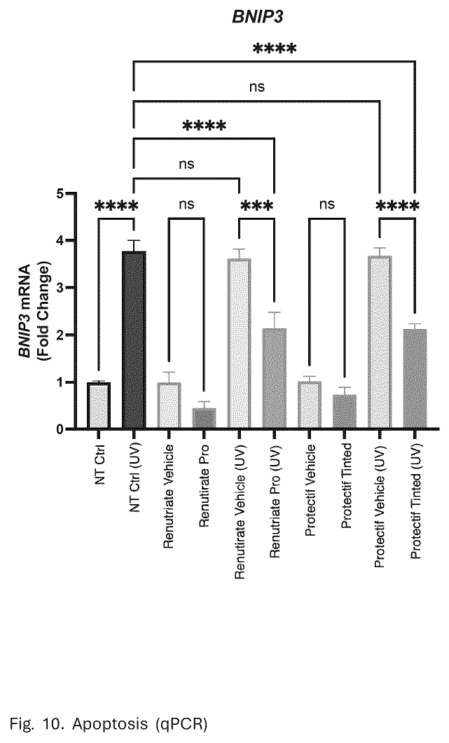

As further described in FIG. 10, epidermal cells were treated with cream products containing the Product Ingredients or the Vehicle and tested in the presence or absence of UV radiation (stress). The Product Ingredients in Renutriate PRO and Protectif Tinted significantly decreased the production of BNIP3 mRNA under stress conditions (e.g., UV exposure) compared to Vehicle. BNIP3 expression is associated with cellular senescence and apoptosis. BNIP3 regulates programmed cell death (apoptosis) and is involved in cellular stress responses. Reduction of BNIP3 by Product Ingredients delays cell death, prolonging the lifespan of a healthy epidermal cells and ensuing an overall improved skin function. Delayed apoptosis prevents premature cell death, a hallmark of cellular senescence. Products containing Product Ingredients ensures that skin cells can continue to function optimally throughout the cell cycle.

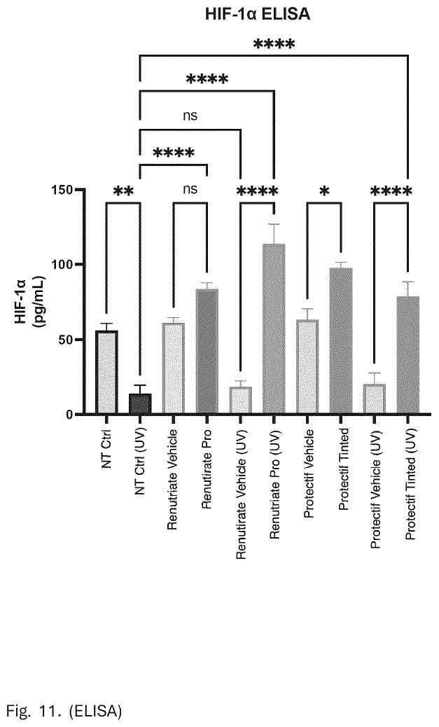

As further described in FIG. 11, epidermal cells were treated with cream products containing the Product Ingredients or the Vehicle and tested in the presence or absence of UV radiation (stress). The Product Ingredients in Renutriate PRO and Protectif Tinted significantly increased the production of HIF-1α protein production under naïve and stress conditions (e.g., UV exposure) compared to Vehicle. Increase in HIF-1α protein expression promotes angiogenesis and skin oxygenation. HIF-1α is a transcription factor that responds to low oxygen levels by promoting the formation of new blood vessels (angiogenesis). Increase in HIF-1α protein by Product Ingredients improves oxygen delivery and nutritional flow to epidermal cells, facilitating better healing and regeneration. Angiogenesis is essential for maintaining skin vitality. Enhancement of HIF-1α addresses mitochondrial dysfunction and loss of nutrient sensing, improving overall skin health.

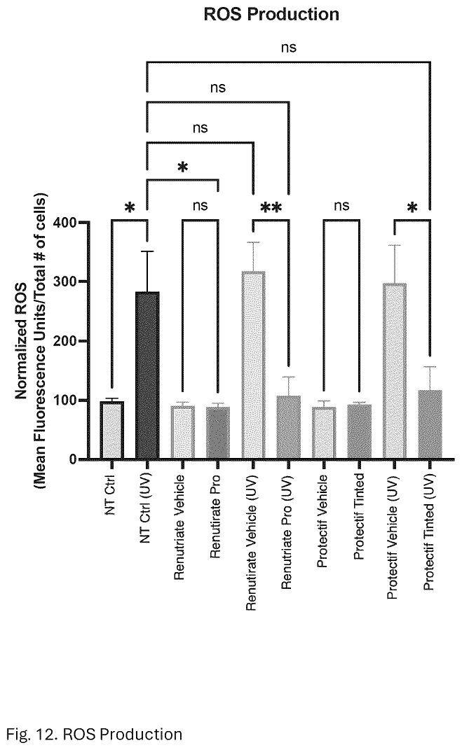

As further described in FIG. 12, epidermal cells were treated with cream products containing the Product Ingredients or the Vehicle and tested in the presence or absence of UV radiation (stress). Product Ingredients in Renutriate PRO and Protectif Tinted significantly decreased the amount of Reactive oxygen species (ROS) production in epidermal cells under stress conditions (e.g., UV exposure) compared to Vehicle. ROS causes oxidative damage to the cells, a major contributor to DNA damage, pigmentation, and skin aging. Reduction of ROS levels by Product Ingredients prevents DNA damage and significantly reduces the chances of DNA mutations. Additionally, the reduction of ROS levels preserves skin structure. Oxidative stress accelerates aging by damaging DNA and cellular structures. Product Ingredients reduction of ROS directly combats genomic instability and mitochondrial dysfunction, tow critical hallmarks of aging.

The Product Ingredients exhibit a comprehensive mechanism of action that addresses the twelve (12) hallmarks of aging. Through multiple pathways, including but not limited to: collagen synthesis, ECM remodeling, reduction in inflammation, stress response modulation, and angiogenesis promotion. The combination of increased collagen production. The combination of increased collagen production, enhanced ECM stability, reduced inflammatory markers, and improved stress resilience suggests that Product Ingredients restores and maintains skin health.

### Twelve Hallmarks of Aging

Product Ingredients help alleviate the twelve hallmarks of aging. Protein Ingredients reduce loss of proteostasis by significantly increasing collagen synthesis and boosting DCN expression. In doing so, Product Ingredients improve firmness of the skin, thereby reducing wrinkles and maintaining balance in the skin's extracellular matrix (ECM). Protein Ingredients upregulate the protein production of LAMB1, combating cellular senescence and maintaining nuclear integrity. Protein Ingredients reduce BNIP3 expression, delaying programmed cell death and thus extending cell lifespan which preserves youthful appearance of the skin. Reduction of ROS levels in stress condition allows Protein Ingredients to minimize DNA damage to extend cell lifespan. Furthermore, Protein Ingredients increases DDIT4 expression, enhancing DNA repair mechanisms protecting cellular DNA against mutations thereby increasing genomic integrity. Protein Ingredients also enhance LAMB1, LAMB4 and LAMA4 which support nuclear architecture which indirectly influences telomere stability. The preservation of telomere length promotes cell longevity. Protein Ingredients have been shown lower ROS levels, thereby protecting mitochondria from oxidative stress. Additionally, Protein Ingredients modulate BNIP3 in the epidermal cells, thereby influencing mitochondrial turnover and mitochondrial-related aging.

Protein Ingredients have also been shown to increase DDIT4 transcription, an integral component in regulating metabolic pathway under stress thereby providing improved cellular metabolism. By enhancing ECM components, Protein Ingredients were able to create supportive environment in the epidermis for stem cell functionality and improved tissue repair. Protein Ingredients reduce pro-inflammatory cytokines IL-6 and IL-13 and boost HIF-1α and VEGF thereby restoring cell signaling and improved tissue homeostasis while reducing inflammation. Protein Ingredients modulates BNIP3 which affects reduces autophagy, thereby enhancing cellular cleanup process, maintaining cellular health.

Protein Ingredients have been shown to do the following: 1. Enhance skin structure and integrity; mitigate chronic inflammation; 2. improving cellular resilience and repair; and 3. boosting skin healing and radiance. Through collagen synthesis and ECM remodeling (via P4H1, LAMB1, LAMB4, LAMA4 and DCN) the Protein Ingredients strengthen the skin's structural components, preventing sagging, wrinkling, and loss of elasticity. Protein Ingredients reduced inflammatory cytokines (IL-6, IL-13) thereby preventing chronic inflammation, a leading cause of aging via tissue breakdown and degradation. By enhancing stress response pathways (DDIT4 and HIF-1α) Protein Ingredients are able to promote angiogenesis, enabling skin cells to better withstand environmental stressors like UV damage and oxidative stress. The further promotion of angiogenesis (VEGF and HIF-1α) allows Protein Ingredients to improve blood flow and oxygenation in the epidermis, ensuring that skin cells receive the nutrients needed for repair and rejuvenation, leading to improved skin texture and radiance. Protein Ingredient's multi-faceted approach to skincare addresses the core biological processes involved in aging. Through responding to the skin's needs, Protein Ingredients offers and advanced, adaptive solution for comprehensive anti-aging Topical Skin Composition product.

### Tissue Repair

The use of products containing Product Ingredients substantially increased VEGF expression under stress (UV-exposure). VEGF, or vascular endothelial growth factor, plays a crucial role in angiogenesis and tissue repair, facilitating the formulation of new blood vessels and enhancing nutrient delivery to damaged tissues. Protein Ingredients not only protects but also actively enhances the skin's regenerative capacity, a critical factor in preventing and repairing UV-induced damage.

### Activation of Stress-response Pathways

The use of products containing Product Ingredients lead to the upregulation of key molecular markers associated with cellular stress response and cellular survival. Protein Ingredients increased the expression of DDIT4, HIF-1α and P4H1, which are essential in mediating responses to hypoxia and oxidative stress. HIF-1α, a master regulator of cellular response to low oxygen conditions, is particularly important in maintaining cellular homeostasis and promoting survival under stress. The upregulation of expression of DDIT4, HIF-1α and P4H1 enhances the skin's intrinsic repair mechanisms, reducing the long-term effects of UV-induces DNA damage.

### Cellular Structural Integrity

The use of products containing Product Ingredients lead to the upregulation of LAMB1, which is critical component of the nuclear lamina, essential for maintaining nuclear structure and function. Profiling of epidermal cells has identified downregulation of LAMB1 as a biomarker of cellular senescence. Cellular senescence is implicated for tissue aging and loss of function. Product Ingredients upregulates LAMB1 which not only prevents (or delays) the onset of cellular senescence but also acts as a senolytic agent, thereby promoting long-term skin health and resilience. Product Ingredients have shown to enhance regenerative capacity of the skin.

### Carcinogenesis

The use of products containing Product Ingredients reduces or eliminates inflammation and oxidative stress, key drivers of aging and carcinogenesis. The continued application of products containing Product Ingredients exert a robust anti-inflammatory effect, as evidenced by significant reduction of pro-inflammatory cytokines IL-6 and IL-1b. Furthermore, the use of products in Product Ingredients on the skin normalizes ROS levels in presence in UV exposure. The reduction of ROS and inflammatory markers underscores the potential of Product Ingredients to prevent chronic inflammation, a known contributor to premature skin aging and cancer development.

### Extracellular Matrix

The use of products containing Product Ingredients supports the maintenance and repair of the extracellular matrix, contributing to structural integrity and function of the skin. UV exposure typically results in drastic reduction of DCN levels, leading to impaired collagen fibrillogenesis and compromised skin structure. The use of products containing Product Ingredients following UV exposure increased the DCN expression.

### Rosacea and Atopic Dermatitis

Sensitive skin conditions like rosacea and atopic dermatitis can manifest as redness, inflammation, and varying degrees of discomfort due to a compromised epidermis. Rosacea is typically marked by persistent redness, inflammatory papules, and a tendency towards flare-ups triggered by external factors (such as environment). Atopic dermatitis is characterized by red, itchy patches of dry, flaking skin. Both conditions often present challenges in treatment, which is further exacerbated with inflammation.

### Post-inflammatory Hyperpigmentation

Post-inflammatory hyperpigmentation (“PIH”) varies in severity based on the underlying causes, such as psoriasis, lichen planus, or acne. Each condition presents unique challenges due to differences in the inflammatory processes and the resultant skin response.

Product Ingredients have been shown to be effective in reducing PIH irrespective of the underlying causes. Renutriate was shown to significantly reduce hyperpigmented lesions with marked improvements in skin texture and discoloration. After sixteen (16) weeks of continuous treatment with Renutriate, HIP is minimal and, in some cases, completely cleared.

### Patient Example 1

As further described in FIGS. 13A to 13E, the head of Patient 1 is shown. Patient 1 applied Renutriate PRO product containing the Product Ingredients twice (2) a day for eight (8) months. With specific reference to FIG. 13A, the figure shows the patient's skin at day zero (0). Patient 1's skin prior to the application of Renutriate PRO contained the following conditions: Actinic Keratosis, Seborrheic Keratosis, xerosis and photodamage. These conditions have the potential to cause the following diseases: Squamous Cell Carcinoma, Basal Cell Carcinoma, pruritus, seborrheic dermatitis. Over time, and with continued use of Renutriate PRO, Patient 1's skin shows improvements in almost complete resolution of Actinic Keratosis, Seborrheic Keratosis, xerosis and photodamage and with increased radiance and improved texture, with a smoother, more even skin. With Specific reference to FIG. 13B, Patient 1's skin shows a marked improvement after sixty (60) days of continued application of Renutriate PRO containing the Product Ingredients. The skin shows a decreased of Actinic Keratosis, Seborrheic Keratosis, and xerosis and photodamage. With Specific reference to FIG. 13C, Patient 1's skin shows additional healing and a further loss of Actinic Keratosis, Seborrheic Keratosis, xerosis and photodamage after one hundred and twenty (120) days of continued application of Renutriate PRO containing the Product Ingredients. With Specific reference to FIG. 13D, Patient 1's skin shows further healing and rejuvenation. This skin looks enhanced, healthier and younger with marked improvement in skin radiance. With specific reference to FIG. 13E, Patient 1's skin displays a rejuvenated and healthy appearance after two-hundred and forty (240) days of continued application of Renutriate PRO containing the Product Ingredients. The skin shows an almost complete resolution of Actinic Keratosis, Seborrheic Keratosis, xerosis and photodamage. Throughout Patient 1's use of Renutriate PRO containing the Product Ingredients, the Patient 1 skin displays increased collagen production and ECM remodelling which has led to enhanced skin texture and luminosity. Furthermore, improved angiogenesis boots microcirculation further enhancing skin radiance.

### Patient Example 2

As further described in FIGS. 14A to 14C, the head of Patient 2 is shown. Patient 2 applied Renutriate PRO product containing the Product Ingredients twice (2) a day for four (8) months. With specific reference to FIG. 14A, the figure shows the patient's skin at day zero (0). Patient 2's skin prior to the application of Renutriate PRO exhibited the following conditions:

Actinic Keratosis, Seborrheic Keratosis, photodamage and wrinkles. These conditions have the potential to cause the following diseases: Squamous Cell Carcinoma, Basal Cell Carcinoma, pruritus, photodamage and loss of elasticity and firmness. Over time, and continued use of Renutriate PRO, Patient 2's skin shows improvements in radiance and texture, with a smoother, more uniform skin. With specific reference to FIG. 14B, the Patient 2's skin looks healthy with reduced pitting and redness after sixty (60) days of continued use of Renutriate PRO. With specific reference to FIG. 14C, the Patient 2's skin shows improved skin stasis and a healthier, rejuvenated look after one hundred and twenty (120) days of continued use of Renutriate PRO. Patient 2's skin shows a significant reduction in wrinkle depth and reduction in fine lines.

Enhanced collagen synthesis by Renutriate PRO through increased production of P4H1 and DCN expression contributes to firmer, smoother skin. The resultant effect is seen in FIG. 14C, smoother skin reducing the appearance of wrinkles.

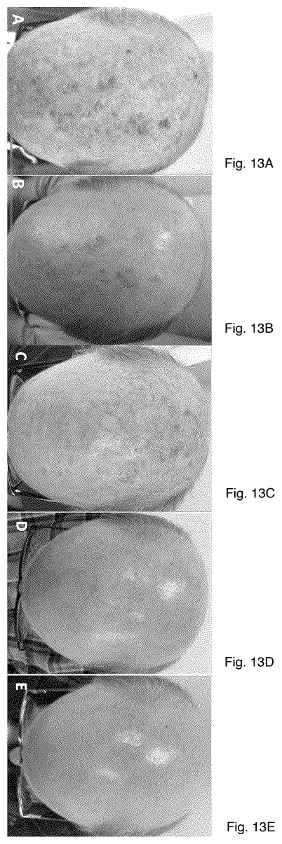

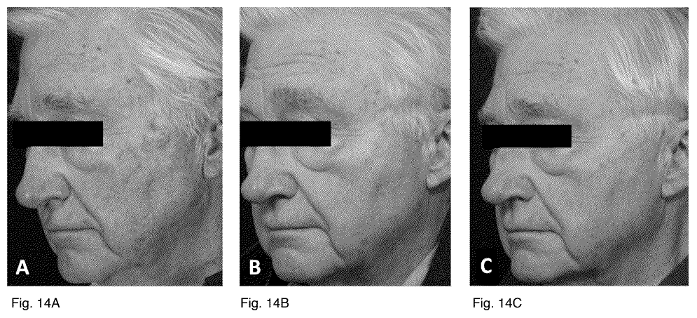

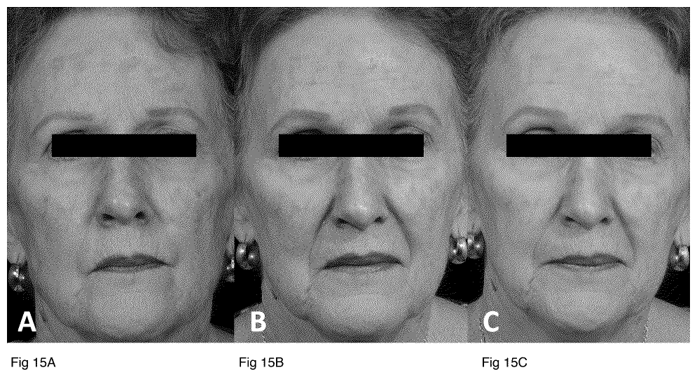

As further described in FIGS. 15A to 15C, the head of Patient 3 is shown. Patient 3 applied Renutriate PRO product containing the Product Ingredients twice (2) a day for four (4) months. With specific reference to FIG. 15A, the figure shows the patient's skin at day zero (0). Patient 3's skin prior to the application of Renutriate PRO exhibited the following conditions: Actinic Keratosis, Seborrheic Keratosis, photodamage and wrinkles. These conditions have the potential to cause the following diseases: Squamous Cell Carcinoma, Basal Cell Carcinoma, pruritus, photodamage and loss of elasticity and firmness. Over time, and continued use of Renutriate PRO, Patient 3's skin shows reduced signs of inflammation and a significant reduction of redness. With specific reference to FIG. 15B, the Patient 3's skin shows decreased signs of inflammation and Actinic Keratosis and photodamage after sixty (60) days of continued use of Renutriate PRO. Patient 3 shows significant increase in skin firmness and elasticity. With specific reference to FIG. 15C, the Patient 3's skin is shown after hundred and twenty (120) days of continued use of Renutriate Pro. Patient 3's skin shows a dramatic reduction in inflammation and redness. The patient's skin, after continued use of Renutriate PRO is markedly firmer with reduced skin sagging. The reduction of IL-6 and IL-16 demonstrates Renutriate PRO's anti-inflammatory properties, resulting in calmer, less irritated skin.

### Patient Example 4

As further described in FIGS. 16A and 16B, the head of Patient 4 is shown. Patient 4 applied Renutriate PRO product containing the Product Ingredients twice (2) a day for two (2) months. With specific reference to FIG. 16A, the figure shows the patient's skin at day zero (0). Patient 4's skin prior to the application of Renutriate PRO exhibited the following conditions Actinic Keratosis, photodamage and severe rhytids. These conditions have the potential to cause the following diseases: Squamous Cell Carinoma, Basal Cell Carcinoma, pruritus, photodamage fand loss of elasticity and firmness, and flaking and dryness. Over time, and continued use of Renutriate PRO, Patient 4's skin shows reduced signs of redness and wrinkles. With specific reference for FIG. 16B, Patient 4's skin shows a progressive improvement in skin elasticity and firmness. Upon repeated use of Renutriate PRO, the patient's skin shows enhanced ECM remodeling via LAMB1 and LAMB4 modulation and restored skin firmness. The patient's skin also shows improved overall skin health and vitality with dramatically reduced depth of wrinkles.

### Patient Example 5

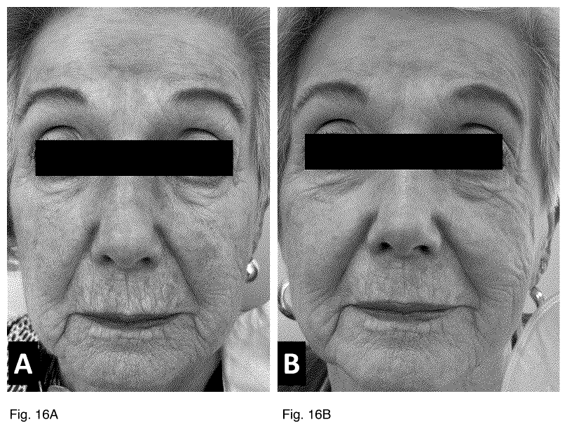

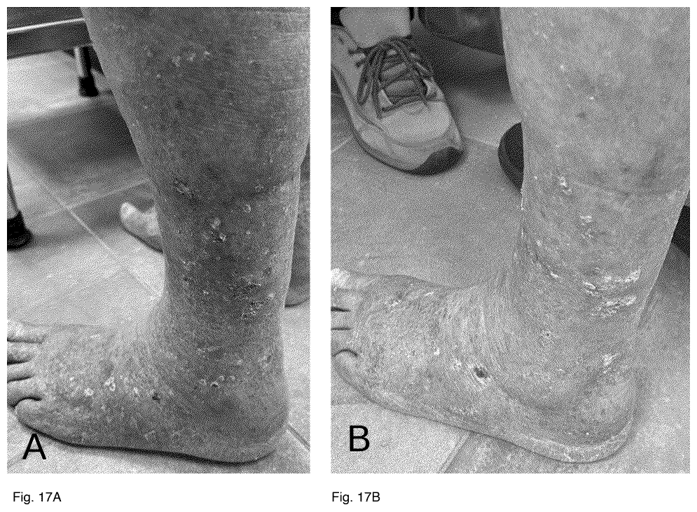

As further described in FIGS. 17A and 17B, the leg of Patient 5 is shown. Patient 5 applied Renutriate PRO product containing the Product Ingredients twice (2) a day for two (2) months. With specific reference to FIG. 17A, the figure shows the patient's leg at day zero (0). Patient 5's skin prior to the application of Renutriate PRO exhibited the following conditions: Actinic Keratosis, Seborrheic Keratosis, stucco keratoses, retention keratoses, hypertrophic actinic keratosis, Bowen's disease, Squamous Cell Carcinoma, edema, and venous stasis dermatitis. These conditions have the potential to cause the following diseases: Squamous Cell Carcinoma, Basal Cell Carcinoma, pruritus, photodamage and loss of elasticity and firmness, flaking and dryness, venous stasis ulcers, comprised blood flow, ischemia. Over time, and continued use of Renutriate PRO, Patient 5's skin showed reduced sings of Actinic Keratosis, Seborrheic Keratosis, stucco keratoses, retention keratoses, Hypertrophic actinic keratosis, Bowen's disease, Squamous Cell Carcinoma, edema, and venous stasis dermatitis. With specific reference to FIG. 17B, Patient 5's skin exhibited reduced pigmentation and dark spots, leading to a more even skin tone. The amount and severity of Actinic Keratosis, Hypertrophic Actinic Keratosis, Seborrheic Keratosis, and Stucco Keratosis are markedly and significantly reduced. The patient's skin also shows significant reduction in inflammation and skin dryness/flaking and edema. Upon continued application of Renutriate PRO, the patient skin shows a reduces levels of ROS and enhanced DNA repair mechanisms as seen by a marked reduction in pigmentation. Furthermore, Renutriate PRO enhances DNA damage repair with visibly lower burden of Actinic Keratosis. By all accounts all aspects of skin health were improved with continuous use of Renutriate PRO.

### Patient Example 6

As further described in FIGS. 18A and 18B, the back of Patient 6 is shown. Patient 6 had a moderately server sun burn as a result of excessive UV exposure. Post sunburn, Patient 6 applied Renutriate PRO containing Product Ingredients to the left shoulder (as shown specifically in FIG. 18A) twice (2) a day for fourteen (14) days. Post sunburn, Patient 6 applied the leading brand moisturizer to the right-side shoulder (as shown specifically in FIG. 18B) twice (2) a day for fourteen (14) days. The left shoulder showed significant recovery to the sunburn when compared to the right shoulder. The left shoulder treated with Renutriate PRO had rapid recovery from sunburn with reduced redness, pigmentation and skin peeling. The recovery seen in on the left shoulder was as a result of synergy between antioxidant protection (ROS reduction), anti-inflammatory effects (reduction of IL-6 and IL-13), and angiogenesis (VEGF and HIF-1α) which accelerates skin healing and promotes rapid recovery and protection against UV damage.

### Patient Example 7

As further described in FIGS. 19A to 19C, the face of Patient 7 is shown. Patient 7 has presented with a seven (7) year history of moderate rosacea primarily affecting cheeks and chin area. Patient 7 experienced intermittent flares of redness, papules, and inflammatory bumps. The intermittent symptoms are further aggravated by sun exposure and stress. Patient 7 applied Renutriate product containing the Product Ingredients twice (2) a day for sixteen (16) weeks. With specific reference to FIG. 19A, the figure shows the patient's face at day zero (0). Patient 7's skin prior to the application of Renutriate exhibited the following conditions: redness, papules, and inflammatory bumps. Over time, and continued use of Renutriate, Patient 7's skin showed reduced sings of rosacea. With specific reference to FIG. 19B, upon eight (8) weeks of continued application of Renutriate, Patient 7's skin exhibited reduced redness and dark spots, leading to a more even skin tone. The amount and severity of rosacea was significantly reduced. The patient's skin also shows significant reduction in papules and inflammatory bumps. With specific reference to FIG. 19C, the figure shows the patient's face at sixteen (16) weeks. Upon continued application of Renutriate, the patient skin shows a reduced level of rosacea as seen by a marked reduction in inflammation, papules and redness. The patient's skin exhibited a significantly calmer and smoother appearance, with residual redness only faintly visible. The Product Ingredients of Renutriate were able to reduce the symptoms of rosacea through the modulation and downregulation of inflammatory cytokines such as IL-13 IL-6 and CCL2 which are critical for induction of rosacea, and which have been shown to be reduced by Product Ingredients.

### Patient Example 8

As further described in FIGS. 20A to 20C, the hand of Patient 8 is shown. Patient 8 has presented with a two (2) year history of moderate atopic dermatitis primarily the hands.

Patient 8 experienced intermittent flares ups of during winter seasons, leading to cracked, peeling skin. The intermittent symptoms are presented with itchy, dry, and inflamed. Patient 8 applied Renutriate product containing the Product Ingredients twice (2) a day for two (2) weeks. With specific reference to FIG. 20A, the figure shows the patient's face at day zero (0). Patient 7's skin prior to the application of Renutriate exhibited the following conditions: itchy, cracked, and dry skin around the knuckles, finger joints, and finger webbing. Over time, and continued use of Renutriate, Patient 8's skin showed reduced sings of atopic dermatitis. With specific reference to FIG. 20B, upon one (1) week of continued application of Renutriate, Patient 8's skin exhibited reduced severity of redness and flakiness. The itching sensation significantly subsided, and the visible cracks in the skin has become to heal. With specific reference to FIG. 20C, the figure shows the patient's hand at two (2) weeks. Upon continued application of Renutriate, the patient skin shows further resolution of dryness and inflammation. The patient's skin exhibited a significantly calmer appearance, with only minimal residual redness and scaling. The Product Ingredients of Renutriate were able to reduce the symptoms of atopic dermatitis through the modulation of the inflammatory pathway likely through the downregulation of the CCL-2 -CCR2 pathway.

### Patient Example 9

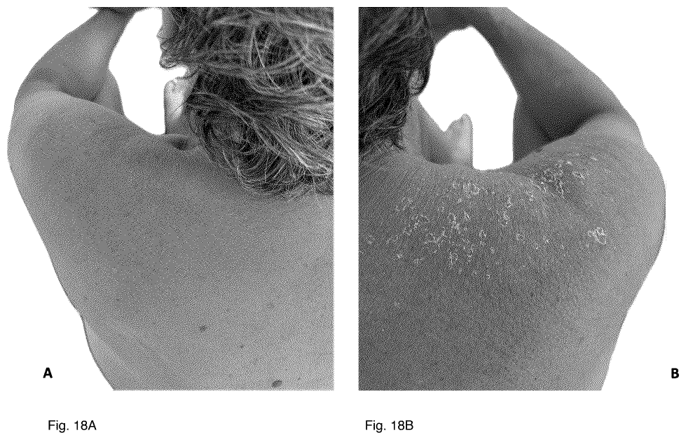

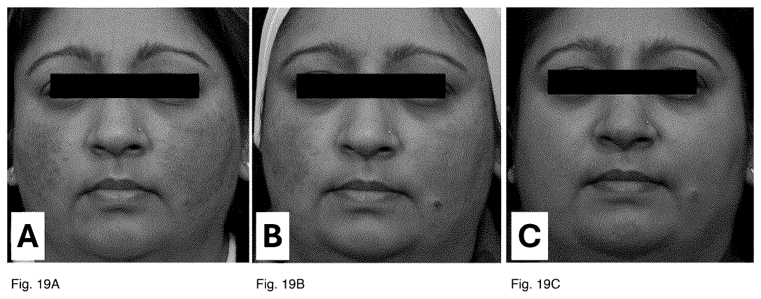

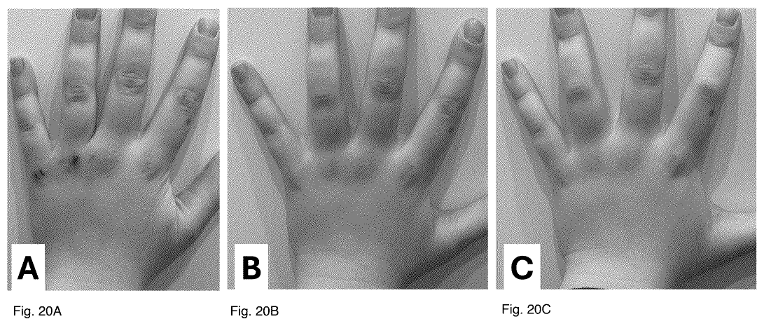

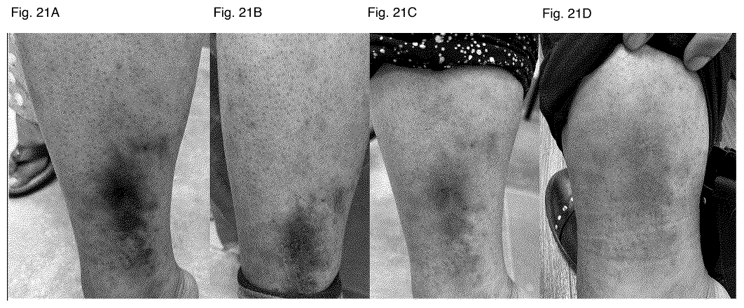

As further described in FIGS. 21A to 21D, the ankle of Patient 9 is shown. Patient 9 has presented with a ten (10) year history of psoriasis primarily in the lower extremities.

Patient 9 applied Renutriate product containing the Product Ingredients twice (2) a day for sixteen (16) weeks. With specific reference to FIG. 21A, the figure shows the patient's ankle at day zero (0). Patient 9's skin prior to the application of Renutriate exhibited a well-demarcated hyperpigmented patches that contained a dark brown to grayish-brown colour. With specific reference to FIG. 21B, upon eight (8) weeks of continued application of Renutriate, Patient 9's skin exhibited a significant reduction in hyperpigmentation. The surrounding skin exhibited reduced flakiness and a much smoother appearance. With specific refence to FIG. 21C, the figure shows the Patient 9 skin at twelve (12) weeks. The patient's skin shows even more reduction in the hyperpigmentation with a more even skin. The skin appeared to have more skin elasticity with an overall smoother feel. With specific reference to FIG. 21D, the figure shows the Patient 9 skin at sixteen (16) weeks of continued use of Renutriate. The patient's skin has minimal pigmentation and an almost complete resolution of the hyperpigmented patches. The patient's skin appeared healthy and well balanced.

### Patient Example 10

As further described in FIGS. 22A to 22C, the hands of Patient 10 are shown. Patient 10 has presented with a five (5) year history of lichen planus affecting her hands, neck, and chest. Patient 10 applied Renutriate product containing the Product Ingredients twice (2) a day for sixteen (16) weeks. With specific reference to FIG. 20A, the figure shows the patient's hands at day zero (0). Patient 10's skin prior to the application of Renutriate exhibited a well-demarcated hyperpigmented patching that contained a grayish-brown colour. Over time, and continued use of Renutriate, Patient 10's skin showed marked reduction in hyperpigmentation. With specific reference to FIG. 22B, the patient's hands are shown upon eight (8) weeks of continued use of Renutriate. The patient's hands exhibited a significant reduction in hyperpigmentation. The surrounding skin exhibited reduced flakiness and a much smoother appearance. With specific refence to FIG. 22C, the figure shows the Patient 9 skin after sixteen (16) weeks continued use of Renutriate. The patient's skin has minimal pigmentation and an almost complete resolution of the hyperpigmented patches. The patient's skin appeared healthy and well balanced.

### Patient Example 11

As further described in FIGS. 23A to 23C, the face of Patient 11 is shown. Patient 11 has presented with a one (1) year history of acne vulgaris on her face. Patient 11 applied Renutriate PRO product containing the Product Ingredients twice (2) a day for sixteen (16) weeks. With specific reference to FIG. 23A, the figure shows the patient's face at day zero (0). Patient 11's skin prior to the application of Renutriate exhibited acne and well-demarcated hyperpigmentation located around previous acne lesions. Over time, and continued use of Renutriate, Patient 11's skin showed reduced sings of acne and hyperpigmentation. With specific reference to FIG. 23B, upon eight (8) weeks of continued application of Renutriate PRO, Patient 11's skin exhibited reduced severity of acne and hyperpigmentation. With specific reference to FIG. 23C, the figure shows the patient's face at sixteen (16) weeks. Upon continued application of Renutriate PRO, the patient skin shows further resolution of acne and hyperpigmentation. The patient's skin exhibited minimal acne lesions with an almost completely cleared skin. The skin looked healthier and more vibrant. Additionally, the patient's skin has minimal pigmentation and almost complete resolution of the hyperpigmented patches. The Product Ingredients of Renutriate were able to reduce the symptoms of acne and hyperpigmentation.

### Reversing the Aging Process

The Renutriate PRO and the Protectif Tinted products, when applied to the skin have shown to not only reduce the rate of premature cellular aging but have also shown to reverse the aging process. In vitro and in vivo analysis of epidermal cells coated with Renutriate PRO or Protectif Tinted have shown dramatic results with respect to a number of key cellular components.

As described in FIGS. 6, 7, and 11, epidermal cells coated with Renutriate PRO and Protectif Tinted have shown dramatic effects on the key cellular components. FIGS. 6, 7, and 11 describe the modulation of expression of key cellular components in response to environmental stressors. Epidermal cells coated with Renutriate PRO and Protectif Tinted show a ⅔ reduction in the expression of IL6 (FIG. 6), a one-half reduction in IL-16 (FIG. 7), and a 5-fold increase in HIF-1α (FIG. 11) when exposed to environmental stressors. As key cellular components expressed as a result of environmental stressors, IL6, IL-13, and HIF-1α also play a pivotal role in premature aging. Environmental stressors induce the modulation of key cellular components within the cell, which in turn age the cell prematurely.

Oxidative stress is another environmental stressor that plays an essential role in premature aging. Effects of oxidative stress, and the resultant formation of ROS, have a detrimental effect on the skin, and has been shown to be a key contributor to cellular senescence. ROS is also shown to significantly increase the rate of premature aging of the skin. As further described in FIG. 12, the epidermal cells incubated with the Renutriate PRO have a 3-fold decrease in the production of ROS in the presence of environmental stressors. The reduction was so significant that the ROS production was returned to the naïve levels (without presence of environmental stressors).

The Renutirate PRO has been tested on numerous patients and has shown a dramatic modulation of the expression of key cellular components as a result of environmental stressors. Such a response to environmental stressors allows the epidermal cell to not only effectively combat the effects of the environmental stressors but also significantly reduce the rate of premature aging. As a result, it has been shown that the use of Renutriate PRO decreases the overall age of the skin.

### Patient Example 12

Patient 12, a 47-year-old male has been actively applying Renutriate PRO and Protectif Fair Tint, once daily for nine (9) months on the left forearm. Patient 12 has also been actively applying a leading brand skin care product (moisturizer and sunscreen), once daily for nine (9) months on the right forearm. Patient 12 leads a fairly active lifestyle and both forearms are regularly exposed to the environmental stressors. Patient 12's left, and right forearms were subsequently tested for relative age, which is directly correlated to the effect of environmental stressors on premature aging of the epidermal cells. Skin age was tested through a validated PAOT skin scanning devices (the “PAOT”). PAOT is a clinically validated tool that can test, in real time, the oxidative stress and presence of ROS in epidermal cells. PAOT can provide the true age of the skin. In addition to assessing the skins oxidative stress, PAOT tests the presence of: a. Vitamin C Ascorbic acid; b. skin hydration; c. pH balance; d. glutathione GSH; and, e. Vitamin E tocopherol. With specific reference to FIG. 24A, it was determined that Patient 12's left forearm (treated with Renutriate PRO and Protectif Fair Tint) skin age was 23.7. With Specific reference to FIG. 24B, Patient 12's right forearm (treated with the leading brand skin care product) skin age was 53.8. Application of Renutriate PRO and Protectif Fair Tint has shown not only to induce a significant reduction in premature aging but was also able to reduce the skin age beyond Patient 12's actual age (47 years old). Patient 12 has shown that Renutriate PRO and Protectif Fair Tint can modulate the epidermal cells in such a manner to not only slow down the aging process, but to even reverse it. POAT also displayed differences between the left and right forearms with respect to the presence of vitamin C, vitamin E, hydration, pH balance, and total reduced glutathione. The effects seen on Patient 12's left forearm are not only attributable to the decrease of oxidative stress but to the increased expression of DDIT4 (FIG. 8), a DNA repair enzyme that helps protect epidermal cells from UV induced DNA damage and subsequent mis-production of cellular proteins and enzymes.

The ultrasound images presented in FIG. 24 provide a comparative assessment of skin collagen density and extracellular matrix (ECM) integrity under the two distinct skincare regimens. From a dermatological and scientific perspective, these ultrasound images demonstrate a significant difference in the structural organization and density of the dermal layer. The right panel reveals a comparatively lower density of collagen fibers, with visible fragmentation and reduced uniformity, indicating suboptimal extracellular matrix support. This suggests that while leading brand skin care products (i.e., moisturizer and sunscreen) provide a baseline level of protection, they do not actively stimulate dermal regeneration or enhance collagen integrity in a meaningful way. Conversely, the left panel-representing the skin treated with Renutriate PRO and Protectif Fair Tint-exhibits a marked increase in dermal density, with a more organized and cohesive collagen network. This is evidenced by the enhanced echogenicity (brightness and compactness) of the ultrasound scan, which correlates with greater collagen deposition, improved ECM remodeling, and superior biomechanical properties of the skin. The denser, more uniform structure suggests that Renutriate PRO and Protectif Fair Tint actively promote fibroblast proliferation, collagen synthesis, and ECM stabilization, leading to visible and functional improvements in skin resilience, hydration retention, and overall anti-aging effects. Unlike leading brand skincare products that primarily act as passive barriers, Product Ingredients contained within Renutriate PRO and Protectif Fair Tint actively work at the cellular level to stimulate cellular collagen production, fortify the skin's structural integrity, and accelerate tissue regeneration (skins own repair mechanisms). The remarkable difference in ultrasound imaging highlights the efficacy of Renutriate PRO and Protectif Fair Tint in reversing age-related dermal thinning, enhancing skin firmness, and restoring youthful elasticity.

### Patient Example 13

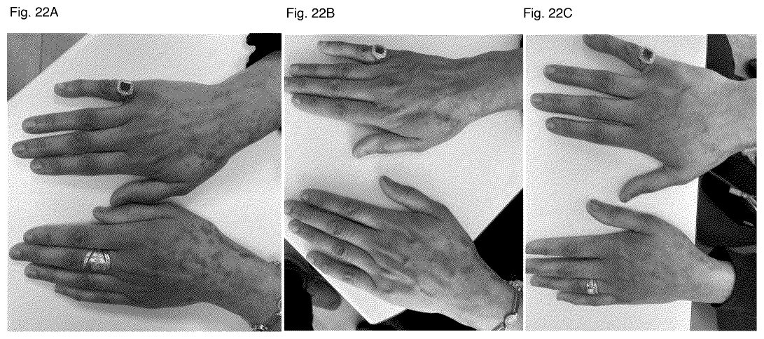

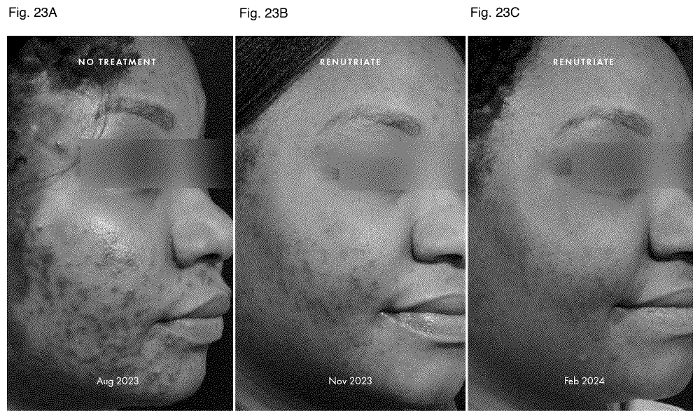

With specific reference to FIG. 25A, Patient 13, a 47-year-old male applied the Renutriate PRO on location 1 and applied the leading brand skin care product on location 2 on the bicep (the “test area”) for twenty (20) minutes. Upon conclusion of the twenty (20) minute skin care incubation period, the test area was cleaned and exposed to UVB stress (UVB lamp Mx1000) for seventy-five (75) seconds. Post UVB stress, location 1 and location 2 were tested for skin age using PAOT. With reference to FIGS. 25B and 25C, PAOT analysis showed that epidermal cells in location 1 were significantly younger that the epidermal cells in location 2. The results indicate that the application of the Renutriate PRO for as little as twenty (20) minutes can significantly impact the regulation of epidermal cellular component expression to reduce the effects of UVB stress exposure and significantly reduce oxidative stress and production of ROS.

### Patient Example 14

Patient 14, a 68-year-old female who has been regularly applying a leading brand skin care product on her face for three (3) years tested her skin age before and after the application of Renutriate PRO. Patient 14 tested her skin using PAOT prior to the application of Renutriate PRO to provide a baseline for her skin age. Patient 14 applied Renutriate PRO on her face and waited for ninety (90) minutes before testing the skin age with PAOT. Patient 14's skin age reduced significantly upon the application of Renutriate PRO. It was shown that application of Renutriate PRO is effective at reducing skin age regardless of the quality and duration of skin care regimen.

In one embodiment, the novel Topical Skin Composition comprises at least one of hydroxytyrosol, green tea extract, and green coffee extract. The concentration of hydroxytyrosol in the Topical Skin Composition can be anywhere from 0.1% w/w to 0.75% w/w. The concentration of green tea extract in the Topical Skin Composition can be anywhere from 0.025% w/w to 0.1% w/w. The concentration of green coffee extract in the Topical Skin Composition can be anywhere from 0.025% w/w to 0.25% w/w. In a preferred embodiment, the green coffee extract can be anywhere from 0.025% w/w to less than 0.2% w/w. In one embodiment, the green coffee extract is green coffee bean extract. The green coffee bean extract may be a Coffee Arabica bean extract. The green tea extract may be a green tea leaf extract, and in one embodiment of the present disclosure, the green tea leaf extract is a Camellia Sinensis leaf extract.

1. A method of activating skin cells, the method comprising:
providing a topical skin composition comprising:
hydroxytyrosol at a concentration range of 0.1% w/w to 0.75% w/w;
green tea extract at a concentration range of 0.025% w/w to 0.1% w/w; and,
green coffee bean extract at a concentration range of 0.025% w/w to less than 0.2% w/w; and
topically applying the topical skin composition to skin.

2. The method of activating skin cells of claim 1, wherein the method of activating skin cells prevents cellular aging of the skin, and wherein the topical skin composition causes at least one of the following:
increases cellular expression of DDIT4 to promote DNA repair in epidermal cells of the skin;
lowers reactive oxygen specie levels and increases production of HIF-1α to increase an oxidative stress response in epidermal cells of the skin;
stimulates cellular expression of P4H1 to enhance collagen biosynthesis in epidermal cells of the skin;
increases cellular expression of at least one of LAMB1, LAMB4, LAMA4, and DCN to increase extracellular matrix remodelling in epidermal cells of the skin;
decreases cellular expression of at least one of IL6 and IL1b to decrease inflammation in response to external stressors;
increases cellular expression of VEGF to increase angiogenesis; and
decreases cellular expression of BNIP3 to reduce apoptosis of epidermal cells of the skin.

3. The method of activating skin cells of claim 1, wherein the method of activating skin cells prevents cellular senescence, and wherein the topical skin composition causes at least one of the following:
increases cellular expression of LAMB1 in epidermal cells of the skin; and, decreases cellular expression of BNIP3.

4. The method of activating skin cells of claim 1, wherein the method of activating skin cells reverses an aging process within skin, and wherein the topical skin composition causes at least one of the following:
increases cellular expression of DDIT4 to promote DNA repair in epidermal cells of the skin;
lowers reactive oxygen specie levels and increases production of HIF-1α to increase oxidative stress response in epidermal cells of the skin; and
decreases cellular expression of at least one of IL6 and IL1b to decrease inflammation in response to external stressors.

5. The method of activating skin cells of claim 1, wherein the topical skin composition is applied twice a day for 14 days to 8 months.

6. The method of activating skin cells of claim 1, wherein the topical skin composition is applied once a day for 9 months.

7. The method of activating skin cells of claim 1, wherein the topical skin composition is applied once for 20 minutes.

8. A topical skin composition comprising:
hydroxytyrosol at a concentration range of 0.1% w/w to 0.75% w/w;
green tea extract at a concentration range of 0.025% w/w to 0.1% w/w; and
green coffee bean extract at a concentration range of 0.025% w/w to less than 0.2% w/w; and
wherein the topical skin composition is applied to a surface of epidermal cells to modulate cellular gene expression profiles essential for skin health, prevention of skin diseases and reduction of severity of affected skin cells.

9. The topical skin composition of claim 8, wherein the topical skin composition increases the total amount of key cellular components that restores skin to its natural healthy state while fortifying natural repair and defence mechanisms of the skin.

10. The topical skin composition of claim 8, wherein topical skin composition counteracts the detrimental effects of environmental stressors, inflammation, and autoimmune disorders.

11. The topical skin composition of claim 8, wherein the modulation of cellular gene expression causes at least one of the following:
reduces IL-6 within the epidermal cells;
increases DDIT4 within the epidermal cells;
increases P4H1 within the epidermal cells;
increases VEGF within the epidermal cells; and
reduces BNIP3 within the epidermal cells.

12. The topical skin composition of claim 8 for the modulation of cellular gene expression to treat various epidermal diseases and reduce symptom severity.

13. The topical skin composition of claim 12, when applied to the surface of skin twice daily, the topical skin composition has the effect of treating at least one of actinic keratosis, seborrheic keratosis, stucco keratosis, retention keratosis, hypertrophic actinic keratosis, xerosis and photodamage.

14. The topical skin composition of claim 12, when applied to the surface of skin twice daily, the topical skin composition the topical skin composition has the effect of treating rosacea.

15. The topical skin composition of claim 12, when applied to the surface of skin twice daily, the topical skin composition has the effect of at least one of treating and reducing the likelihood of at least one of: squamous cell carcinoma and basal cell carcinoma.

16. The topical skin composition of claim 12, when applied to the surface of skin twice daily, the topical skin composition has the effect of treating at least one of: seborrheic dermatitis, venous stasis dermatitis, Bowens disease, edema, venous stasis ulcers, rhytids, comprised blood flow and ischemia, and pruritus.

17. A method of treating one or more of the hallmarks of ageing and skin degradation and collagen and elasticity as a single dosage from a pharmaceutical composition, the method comprising:
providing the pharmaceutical composition comprising:
hydroxytyrosol at a concentration range of 0.1% w/w to 0.75% w/w;
green tea extract at a concentration range of 0.025% w/w to 0.1% w/w; and, green coffee bean extract at a concentration range of 0.025% w/w to less than 0.2% w/w; and
topically applying the pharmaceutical composition to skin.

18. The method of treating of claim 17, wherein the pharmaceutical composition causes at least one of the following:
increases cellular expression of DDIT4 to promote DNA repair in epidermal cells of the skin;
lowers reactive oxygen specie levels and increases production of HIF-1α to increase an oxidative stress response in epidermal cells of the skin;
stimulates cellular expression of P4H1 to enhance collagen biosynthesis in epidermal cells of the skin;
increases cellular expression of at least one of LAMB1, LAMB4, LAMA4, and DCN to increase extracellular matrix remodelling in epidermal cells of the skin;
decreases cellular expression of at least one of IL6 and IL1b to decrease inflammation in response to external stressors;
increases cellular expression of VEGF to increase angiogenesis; and
decreases cellular expression of BNIP3 to reduce apoptosis of epidermal cells of the skin.
